(19) United States
(12) Patent Application Publication
(10) Pub. No.: US 2015/0019250 A1
(43) Pub. Date: Jan. 15, 2015

(54) MANAGER-CONTROLLABLE INTEGRATED USER CONTENT AND SERVICES SYSTEM

(71) Applicant: A-VU MEDIA CORP., Minnetonka, MN (US)

(72) Inventors: John D. Goodman, Minnetonka, MN (US); Reed Zuehlke, Orlando, FL (US)

(21) Appl. No.: 14/495,598

(22) Filed: Sep. 24, 2014

Related U.S. Application Data
(63) Continuation-in-part of application No. 12/777,869, filed on May 11, 2010.
(60) Provisional application No. 61/882,602, filed on Sep. 25, 2013.
(60) Provisional application No. 61/180,636, filed on May 22, 2009.

(51) Int. Cl. G06F 19/00; G06F 21/60; H04L 12/911; H04L 29/08; G06F 3/0481; G06F 3/0484

(52) U.S. Cl. CPC G06F 19/3418; G06F 3/0481; G06F 3/04842; H04L 47/70; H04L 67/12; G06F 21/604
USPC 705/2; 707/783

(57) ABSTRACT

The present disclosure presents user content and service systems that are controllable by one or more remote system managers, which may include one or more primary system managers, and utilized by one or more users, and methods and apparatuses related thereto. For example, the present disclosure presents an example method for multimedia presentation to a user, which may include receiving, at a user terminal, content from a network entity, wherein at least a portion of the content is controllable by a remote manager. Furthermore, such an example method may include displaying the content to the user via a user interface and receiving one or more interactive inputs from the user at the user terminal. Moreover, the example method may include performing one or more functions associated with the content based on the one or more interactive inputs.

### CROSS REFERENCE TO RELATED APPLICATION

This application claims priority to Provisional Application No. 61/882,602, including Appendix A of Provisional Application No. 61/882,602, filed Sep. 25, 2013, and is a continuation-in-part of U.S. application Ser. No. 12/777,869, filed May 11, 2010, which claims priority to Provisional Application No. 61/180,636, filed May 22, 2009, all of which are herein incorporated by reference in their entirety.

### TECHNICAL FIELD

Aspects of the present disclosure relate generally to methods, apparatuses, and systems for providing integrated user content and services to one or more users. Some specific aspects relate to user content and service systems that are controllable by one or more remote system managers.

Also, aspects of the present disclosure relate to communication and media distribution. More particularly, some aspects relate to a system for providing communication and media access to a user and an approved network via a simplified user interface having remotely definable content and menu options.

### BACKGROUND

Older adults and the disabled represent very large but significantly underserved markets for today's broadband based connections and services. Next generation networks are emerging with significant focus on delivering bundles of voice, video, and data (e.g., Internet) services, but these new bundles of services have not been developed for these populations and their unique needs or capabilities. Historical offerings that required adaptation to new technology or self-provisioning that includes installation and user maintenance of system parameters and settings have not been widely adopted by these user groups. Broadband network operators frequently view these segments as low dollar and high maintenance. The lack of connection and/or access to advanced services, multimedia applications, web-based medical services, and home support services creates limits and personal isolation when these technologies could be significant assets to meet the user's unique needs. Thus, there is a need for a comprehensive and user-friendly multimedia system and electronic interface that provides user access and associated permissions corresponding to historically independent services and web-enabled applications and is controllable by one or more system managers for the benefit of the primary user.

Also, access to healthcare and its related costs are personal, state, and national issues that will reach a crisis stage without a revolutionary transformation. Healthcare is facing a perfect storm of aging demographics, expanding medical technology, expanding coverage, entitlement mentality, and slow growth in traditional and personal resources. One solution involves new delivery models that move into home delivered healthcare that extend independent living, improve health and disease management, reduce inpatient days of care, and reduce the demands on traditional medical resources. Older adults and the disabled represent very high cost segments in the medical system and therefore must be part of the solution. However, their current lack of effective broadband connections creates barriers to implementing new healthcare technology and care options that simultaneously improve outcomes and reduce costs.

Additionally, there are currently many other similar needs for simplified system integration with control given to a remote system manager for the benefit of the primary end user. Telemedicine systems are another current example where providers want an integrated system that is easy to use and managed by a remote system manager thereby allowing them to focus on the medical care and not the potentially complex technology.

### SUMMARY

The following presents a summary of the aspects of the present disclosure and are not meant to comprise limiting aspects of the disclosure. These and other aspects of the present disclosure will become more fully understood upon review of the detailed description, which follows.

The present disclosure presents user content and service systems that are controllable by one or more remote system managers, which may include a primary system manager and one or more other system managers arranged in a hierarchy. In some examples, the present disclosure further presents an example method for providing user interaction with unified user content and services system at a user terminal. In some examples, such user interaction may include participating in one or more telemedicine functions associated with the system. Such example methods may include receiving, at a user terminal, content from a network entity, wherein at least a portion of the content is controllable by a remote system manager. The example method may further include displaying the content to the user via a user interface and receiving one or more interactive inputs from the user at the user terminal. Furthermore, such example methods may include performing one or more functions or services associated with the content based on the one or more interactive inputs.

In an additional aspect, the present disclosure presents an example apparatus for content presentation to a user, which may include a content receiving component configured to receive content from a network entity, wherein at least a portion of the content is controllable by a remote system manager. Furthermore, such example apparatuses may include a display component configured to display the content to the user via a user interface and an input receiving component configured to receive one or more interactive inputs from the user at the user terminal. Additionally, the example apparatus may include a user interaction component configured to perform one or more functions or services associated with the content based on the one or more interactive inputs.

Moreover, the present disclosure presents example methods for controlling a user experience associated with user content and services system by a remote manager. Such example methods may include selecting a user of a remote user terminal from a user list, setting content permissions defining the content and services that are accessible to the user, and saving the permissions.

In another aspect, media content and communication services are provided to a user. A schedule of media content and communication services provisioned for the user is received, and an interactive menu is generated including selectable options for each of the provisioned media content and communication services. The interactive menu is stored in a host device associated with the user. The interactive menu is displayed on a display connected to the host device, and the host device receives input signals from a remote control that correspond to selections of the selectable options on the interactive menu. The media content or communication service associated with an option on the interactive menu is initiated when selected with the remote control.

In yet another aspect, a system provides media content and communication services to users. The system includes one or more interactive terminals each associated with a user and including a host device, a display, and a remote control. The system also includes a centralized systems portion in communication with each host device of the one or more interactive terminals. The centralized systems portion, which may include a local or network server, receives a schedule of media content and communication services provisioned for each user and generates an interactive menu specific to the user including selectable options for each of the provisioned media content and communication services. The centralized systems portion also provides the interactive menu specific to the user to the host device of the interactive terminal associated with the user. The remote control generates input signals to the host device that correspond to selections of the selectable options on the interactive menu, and the host initiates the media content or communication service associated with an option on the interactive menu selected with the remote control.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating functions provided by a united user content and services system contemplated by the present disclosure;

FIG. 2 is a block diagram illustrating an example user content and services system of the present disclosure;

FIG. 3 is a block diagram illustrating an example user interaction component of a user terminal as contemplated by the present disclosure;

FIG. 4 is a block diagram illustrating an example system management component of a system manager terminal as contemplated by the present disclosure;

FIG. 5 is a block diagram illustrating an example of a generic computer device of the present disclosure;

FIG. 6 is a flow diagram illustrating an example method executable by a user terminal for presenting unified user content and services system to a user;

FIG. 7 is a block diagram illustrating an example grouping of logical components for presenting the unified user content and services system to a user;

FIG. 8 is a flow diagram illustrating an example method executable by a manager terminal for controlling the user content and services associated with a remote user of the present disclosure; and

FIG. 9 is a block diagram illustrating an example grouping of logical components for controlling the user content and services associated with a remote user of the present disclosure.

FIG. 10 is a block diagram of an embodiment of a system for communication and media distribution to one or more interactive terminals.

FIG. 11 is a front view of a remote control suitable for use with the interactive terminals shown in FIG. 10.

FIG. 12 is a screen view of an exemplary first or primary screen providing access to provisioned services available via the interactive terminal including a presentation of remote suitable for use with the interactive terminal.

FIG. 13 is a screen view provided on the interactive terminal for selecting from available television services on the system.

FIG. 14 is a screen view provided on the interactive terminal for selecting from a list of stored video options to watch.

FIG. 15 is a screen view provided on the interactive terminal for selecting from music options on the system.

FIG. 16 is a screen view provided on the interactive terminal when viewing a new downloaded photo.

FIG. 17 is a screen view provided on the interactive terminal for selecting voice and text message services.

FIG. 18 is a screen view provided on the interactive terminal for selecting from a list of activated websites that can be accessed on the system.

FIG. 19 is a screen view provided on the interactive terminal for selecting from phone options available on the system.

FIG. 20 is a screen view provided on the interactive terminal for selecting from medical or community service options available on the system.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and modules are shown in block diagram form in order to avoid obscuring such concepts.

The present disclosure describes systems, methods, and apparatuses for providing integrated user services and applications to one or more users at a remote location relative to one or more system managers, which may include one or more primary system managers. For example, the present disclosure presents an electronic system including a user interface presented via a user terminal that combines historically independent services and web-enabled applications into a unified and simplified user interface for delivering content to users and is supported by one or more system managers who manage the technology experience and service options of the user, who may reside at a remote location relative to the one or more system managers. Additionally, the system may leverage multiple content and service managers under the oversight of a primary system manager will deliver the benefits and value of technology to users who may not understand or do not have the time or desire to understand technology by integrating all of these various technologies into one simple, flexible end user experience. This integrated system structure with third party system management has other applications including but not limited to telemedicine systems where providers want the integration of otherwise independent applications, a very simple user interface, and a third party with the ability to manage the system for the primary users.

In an aspect, the electronic interface may include content such as, but not limited to, television, movies, music, pictures, video, internet, data searching capability, educational resources, messaging, interactive video communication, voice communication, medical services, telemedicine, scheduled activities, calendars, alerts, reminders, and other interactive media content, as well as services such as, but not limited to, meal delivery services, property maintenance services, laundry services, and the like.

Furthermore, the user terminal may interface with one or more medical devices to facilitate the monitoring of one or more user physiological characteristics. For example, the user system may be configured to interface with a blood glucose monitor, scale, blood pressure monitor, oxygen monitor, or any other peripheral device configured to measure a physiological characteristic of a user. Additionally, the user system may be configured to transmit a reading provided by the one or more medical devices to one or more remote caregivers and/or caregiver systems or devices to inform one or more caregivers of the physiological characteristic of the user.

In an additional aspect, one or more system managers may each utilize a system manager terminal having an interface that allows the one or more system managers to manage the delivery and presentation of user content to the one or more users, who may be located remotely in relation to the one or more system managers. In a non-limiting aspect, a system manager may select one or more services and/or one or more applications that a particular user may be permitted to access and/or utilize. For example, a system manager may set access or content permissions that allow a selected user of the system to view online restaurant menus and order menu items for delivery to the home of the user, order on-demand television or film content for viewing at a user terminal, but may also deny the user's access to web-based music streaming applications and the ability to place a telephone call to landscaping service businesses. These permissions may be transmitted to the user terminal of the user and stored to filter user access to the selected user content and services.

In addition, in some examples, the system manager may be remote caregiver serving in a medical and/or supervisory capacity to the user. As such, the caregiver serving as system manager may utilize the system manager terminal and related software executable thereon to prompt a user, via the user interface at the user terminal, to take a blood glucose reading using a peripheral blood glucose monitor. Further aspects of the integrated user content and services system are described below in relation to the accompanying Figures.

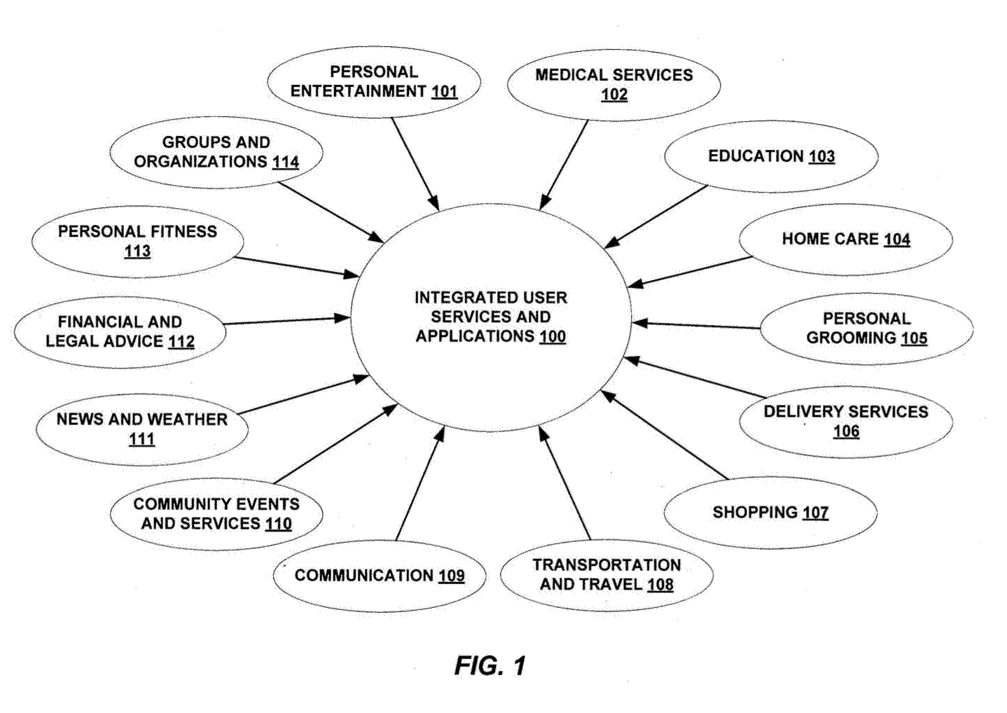

FIG. 1 provides a graphical representation of the myriad applications, services, and other functions that may be consolidated and executed by the presently disclosed unified user content and services system and which a system manager may permit one or more user to access and utilize. As is represented in FIG. 1, several of the example integrated user services and applications 100 available to a user and controllable by one or more system managers of this unified system may include, but are not limited to, personal entertainment 101 (e.g., television and film), medical services (e.g., contacting and/or conducting a virtual appointment with a caregiver), education 103 (e.g. information searching, web searching, information retrieval from the Internet), home care 104 (e.g., handyman or landscaping services), personal grooming 105 (e.g., contacting a barber or stylist), delivery services 106, shopping 107 (e.g., online commerce), transportation/travel 108 (e.g., virtual and otherwise), communication 109 (e.g., voice, video, and/or text communication with one or more contacts, managers or caregivers), community events and services 110, news and weather 111, financial and legal assistance 112, personal fitness 113, groups and organizations 114. These functions are by no means exclusive, and may further include, but are not limited to, time management, personal assistance, meal services, telephone, customer service, sending a caregiver message, taking one more medical readings, viewing a personal health record, ordering medicine, providing information security, calendar, contacts, and managing medical devices. Because each of these functions organized at the periphery of FIG. 1 and those not explicitly shown have typically been performed or provided to a user individually and require intricate, expensive equipment that is sometimes difficult for users to operate, the unified system of user content and services presented in FIG. 1 can significantly improve access to services and the overall multimedia experience for the users of such a system. Additionally, in some examples, one or more telemedicine system functions may be offered to one or more users of the system. In such examples, the telemedicine system and related functions and/or services may include several peripheral medical assessment devices, including, but not limited to, otoscope, stethoscope, exam camera, vitals assessment tools, and the like. Furthermore, a scheduling system may be integrated into the system that can be pre-programmed by the system manager for automated connections, secure video conferencing, access to patient medical records including electronic medical record data, live or recorded imaging, and third parties that can join the consult between the patient and provider.

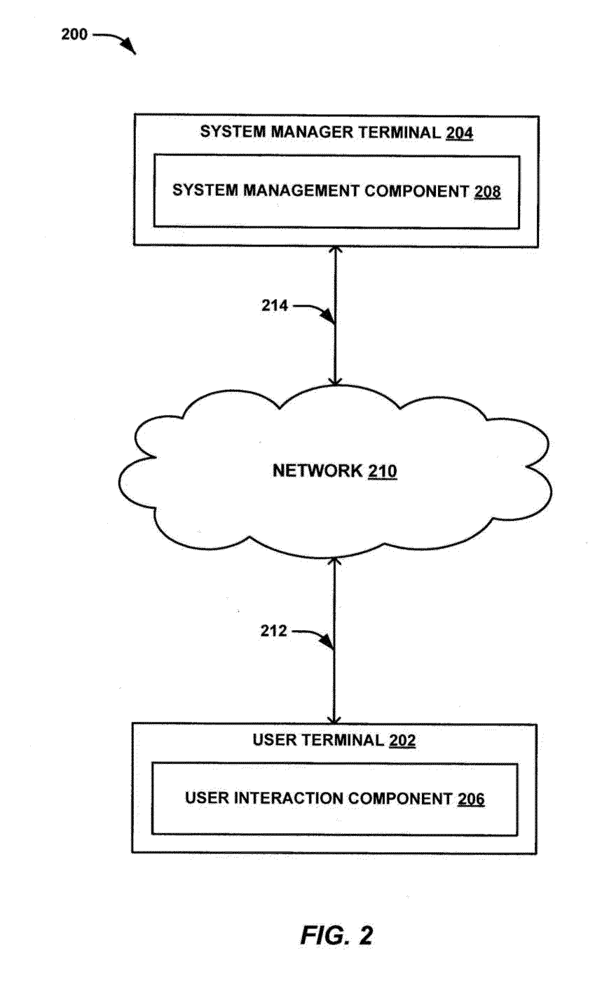

FIG. 2 illustrates an example user content and service system 200 that is configured to provide user content and services to one or more users and to facilitate communication between the one or more users and one or more system managers, who may control user access to such user content and services. Furthermore, in some examples, such system managers may include a hierarchy of system managers. This hierarchy may include a primary system manager and other content or service system managers at a lower level who are granted privileges by the primary system manager to present content and services to the end user. In such examples, the primary system manager may possess overall responsibility for the user experience of one or more users. In other words, the one or more system managers, including the primary system manager, may be to meet one or more user or manager expectations.

As depicted in FIG. 2, system 200 may include one or more user terminals 202, which may be configured to provide the user content and service options to the one or more users and to provide a user interface with which the one or more users can interact. In some examples, the user terminal 202 may include a user interaction component 206 configured to facilitate such interaction. In addition, the one or more user terminals 202 may communicate with one or more system manager terminals 204 and/or receive multimedia and/or medical content via network 210 over communication links 212 and 214. In some examples, the one or more system manager terminals 204 may be associated with one or more primary system managers and one or more lower-level system managers that are under the direction of the one or more primary system managers. In an aspect, system manager terminal 204 may include a system management component 208, which may be configured to manage presentation of a manager interface (e.g. a graphical user interface) to a manager and to control management of one or more user terminals 202 under control of system manager terminal 204. Furthermore, communication links 212 and 214 may be wireless links, wired links, or a combination thereof, and may use one or more communications networks (e.g. network 210) to facilitate this communication and/or multimedia access.

In addition, for purposes of the present disclosure, a system manager may include any person or device that may serve as a manager of the user content and services of one or more users. In some examples, such managers may include one or more third parties charged with managing user content and service permissions associated with one or more users. Alternatively or additionally, the system manager may be any individual, device, system, or group that is charged with undertaking a caregiver role in relation to one or more users or patients. These caregivers may individually include one or more physicians, nurses, family members, parents, hospice caregivers, or the like. For example, in a non-limiting aspect, a user may comprise a medical patient or elderly individual under the care of a hospice caregiver or physician.

Furthermore, communications links 212 and/or 214 may comprise other telecommunication systems and networks, network architectures, and communication standards. By way of example, various aspects may be extended to other Universal Mobile Telecommunications System (UMTS) systems such as Time Division Synchronous Code Division Multiple Access (TD-SCDMA), High Speed Downlink Packet Access (HSDPA), High Speed Uplink Packet Access (HSUPA), High Speed Packet Access Plus (HSPA+) and Time-Division CDMA (TD-CDMA). Various aspects may also be extended to systems employing Long Term Evolution (LTE) (in FDD, TDD, or both modes), LTE-Advanced (LTE-A) (in FDD, TDD, or both modes), CDMA2000, Evolution-Data Optimized (EV-DO), Ultra Mobile Broadband (UMB), Institute of Electrical and Electronics Engineers (IEEE) 802.11 (Wi-Fi), IEEE 802.16 (WiMAX®), IEEE 802.20, Ultra-Wideband (UWB), Bluetooth, and/or other suitable systems. The actual telecommunication standard, network architecture, and/or communication standard employed will depend on the specific application and the overall design constraints imposed on the system 200.

Additionally, network 210 may include an access network and/or core network, and may include any network type, such as, but not limited to, wide-area networks (WAN), wireless networks (e.g. 802.11 or cellular network), the Public Switched Telephone Network (PSTN) network, ad hoc networks, personal area networks (e.g. Bluetooth®) or other combinations or permutations of network protocols and network types. Such network(s) may include a single local area network (LAN) or wide-area network (WAN), or combinations of LANs or WANs, such as the Internet. In addition, network 210 may comprise one or more network devices for storing and delivering multimedia, service, medical content and/or communication services to or between one or more user content and service caregivers included in network 210, system manager terminals 204 and one or more user terminals 202. For example, such network devices may provide user and caregiver authentication services to ensure that such users and/or caregivers are authorized to utilize the system and/or content there. Furthermore, the one or more network devices of network 210 may include one or more storage devices for storing such content, which, in some examples, may include user, system manager, service caregiver, and/or caregiver profiles (including medical reading history data, personal medical record data, identifying data, and the like), custom content associated therewith, and/or executable instructions that, when executed by a processor, may provide the users and/or caregivers with the user interfaces and multimedia and/or medical content. In addition, network 210 may comprise a communication server (e.g. a video or audio conferencing server), which may be configured to manage communication and conferencing between the one or more users and one or more caregivers. Similarly, network 210 may include one or more news and information servers, social networking servers, streaming content servers, voice-over-internet protocol (VoIP) service caregiver devices, messaging service devices, and/or medical data servers configured to facilitate the presentation of multimedia, service, and/or medical content to one or more system manager terminals 204 and one or more user terminals 202.

In addition, network 210 may include one or more of any type of network device, entity, or module, such as an access point, a macro cell, including a base station (BS), node B, eNodeB (eNB), a relay, a peer-to-peer device, an authentication, authorization and accounting (AAA) server, a mobile switching center (MSC), a radio network controller (RNC), or a low-power access point, such as a picocell, femtocell, microcell, etc. Additionally, network entity 104 may communicate with one or more other network entities of wireless, access, fiber-optic, and/or core networks.

Moreover, each of one or more system manager terminals 204 and one or more user terminals 202 may include one or more devices configured to interact with a user or a system manager, respectively, via network 210. In an aspect, the user terminals 202 and/or system manager terminals 204 may each include a personal computer, such as a desktop computer, laptop computer, tablet, set-top box (e.g. connected to a display or television), or other computing device, which may include a processing device or one or more processing modules for executing one or more instructions stored on a computer-readable medium. Furthermore, the one or more user terminals may include one or more mobile devices, which may be known to those skilled in the art as a mobile station, a subscriber station, a mobile unit, a subscriber unit, a wireless unit, a remote unit, a mobile device, a wireless device, a wireless communications device, a remote device, a mobile subscriber station, an access terminal, a mobile terminal, a wireless terminal, a remote terminal, a handset, a terminal, a user agent, a mobile client, a client, or some other suitable terminology.

Furthermore, user terminal 202 and/or system manager terminal 204 may interface with one or more peripheral devices that are configured to, for example, present a user interface (or other output, e.g. sound or haptic feedback) to a user or system manager and/or to obtain input from such a user or system manager. For example, such peripheral devices may include one or more displays, monitors, or the like, one or more speakers, headsets, or the like, and/or one or more haptic feedback devices. Additionally, such a peripheral device may include one or more medical devices to facilitate the monitoring of one or more user physiological characteristics. For example, the user system may be configured to interface with a blood glucose monitor, scale, blood pressure monitor, oxygen monitor, or any other peripheral device configured to measure a physiological characteristic of a user. Additionally, the user terminal 202 may be configured to transmit a reading provided by the one or more medical devices to a network storage device and/or one or more system manager terminals 204 to inform one or more system managers of the physiological characteristic of the user. As such, system 200 may be configured to provide a simple, efficient manager-controllable electronic interface to one or more users that combines historically independent web-enabled services.

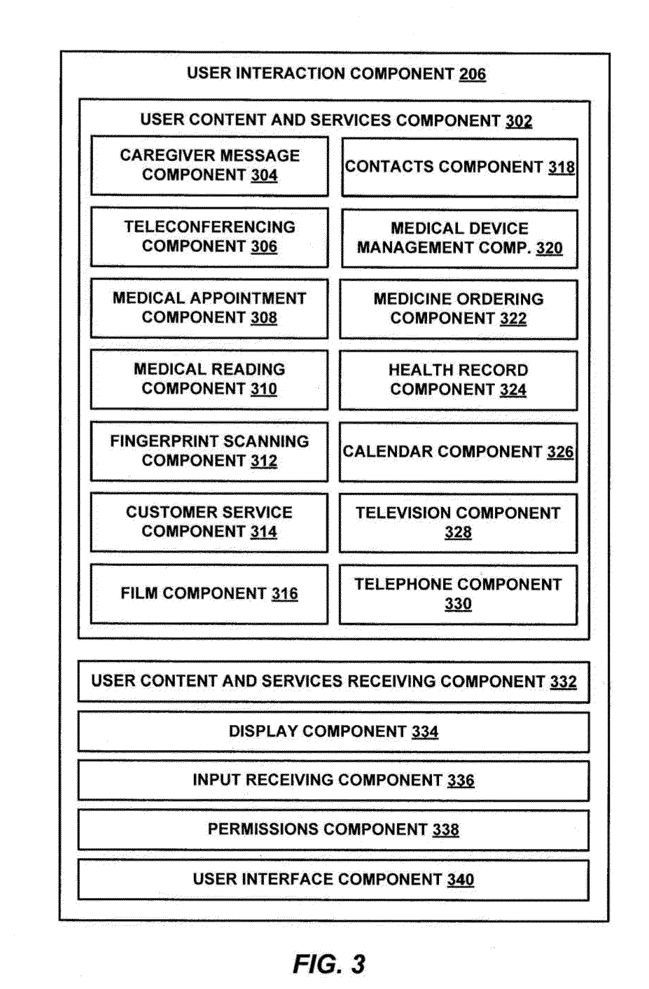

FIG. 3 illustrates several example components of user interaction component 206, introduced in FIG. 2 as a component of user terminal 202, which may be configured to manage interaction between a user and system 200 of FIG. 2. Such interaction may include requesting and/or receiving permissions related to user content and services that may be controlled by one or more system managers of a system manager hierarchy that may include a primary system manager. For example, in an aspect, user interaction component 206 may include a user content and services component 302, which may be configured to manage signals associated with multimedia, service, and/or medical content and control user interaction with a user interface associated with such content. For example, user content and services component 302 may include a component to manage sending a caregiver message 304. In an aspect, sending a caregiver message may include receiving message content input from a user via a peripheral device such as a keyboard and transmitting the message to one or more network entities (e.g. associated with network 210 of FIG. 2) and/or one or caregivers.

Additionally, user content and services component 302 may include a teleconferencing component, which may be configured to initiate, schedule, and/or conduct a teleconference with one or more caregivers. In an aspect, teleconferencing component may be configured to stream real-time video captured by a remote caregiver camera onto a display associated with a user terminal and may further be configured to stream related audio to the user via an attached audio output device (e.g. speakers or headphones). In addition, teleconferencing component 306 may be configured to initiate and manage an outgoing video and/or audio stream comprising a video image and/or voice input of a user captured by an attached camera and/or microphone. Furthermore, teleconferencing component 306 may be configured to synchronize incoming and outgoing teleconferencing content to provide a real-time teleconferencing experience to the user.

In addition, user content and services component 302 may include a medical appointment component 308, which may be configured to facilitate online medical appointments between a user of a user terminal and a caregiver using a caregiver terminal. In an aspect, medical appointment component 310 may allow a user, system manager, and/or a caregiver to initiate such a medical appointment and, in some examples, may manage the medical appointment in a real-time fashion, as described in relation to teleconferencing component 306, above. In addition to allowing such medical appointment teleconference communication, medical appointment component may transmit one or more physiological characteristics associated with the user, which may be embodied in a health record of the user and/or a medical reading, which may be obtained via medical reading component 310.

Such a medical reading component 310 may be included in user content and services component 302, and may be configured to interface with one or more peripheral medical devices or integral user terminal components (not shown), which may be configured to take one or more medical readings of physiological characteristics of the user. For example, medical reading component 310 may be configured to interface with a blood glucose monitor, scale, blood pressure monitor, oxygen monitor, or any other peripheral device configured to measure a physiological characteristic of a user. Additionally, medical reading component 310 may be configured to transmit the medical reading provided by the one or more peripheral medical devices to one or more remote network entities (e.g. physical characteristic database) or caregivers of the physiological characteristic of the user. Furthermore, medical reading component 310 may be configured to receive one or more instructions to a user from a caregiver sent by a caregiver user terminal, which may include an instruction for a user to take a medical reading via the one or more peripheral medical devices.

In an additional aspect, user content and services component 302 may include a fingerprint scanning component 312, which may be configured to bolster information and personal security of a user by requiring the user to scan his or her fingerprint at a fingerprint reader (not shown) before accessing the user content and services. In an alternative aspect, fingerprint scanning component 312 may be replaced by, or user content and services component 302 may additionally contain, a password management component or security component to manage an alternate or additional form of personal information security. Such alternative or additional personal information security methods may include prompting the user to enter a password, passcode, swipe pattern, or any other method of user authentication.

Furthermore, user content and services component 302 may include a customer service component 314, which may be configured to contact and manage user communication with a customer service entity, such as, but not limited to, a customer service server, customer service terminal, or a customer service representative. In an aspect, customer service component 314 may prompt a user for an input, such as, but not limited to a question, communication input, chat message, or the like and may transmit the input to the customer service entity. Moreover, customer service component 314 may be configured to receive one or more communications from a customer service entity, for example, in reply to a previous query or other input to the customer service entity. In some examples, such a customer service entity may include a system manager and/or a caregiver.

In an additional aspect, user content and services component 302 may include a film component 316, which may be configured to receive multimedia film content from one or more multimedia servers and to save such content for subsequent display a user via, for example, a display component 334. In an alternative aspect, film component 316 may display such multimedia film content via display component 334 as streaming content. Furthermore, film component 316 may be configured to provide a film selection interface to the user via display component 334 and to receive one or more film selections from the user corresponding to a film that the user wishes to view. Based on the selection, the film component 316 may then retrieve and display the multimedia film content corresponding to the selection.

In addition, user content and services component 302 may include a contacts component 318 which may be configured to manage one or more contacts associated with a user of the user terminal 202. Contacts component 318 may be configured to allow a user to add a new contact, delete one or more contacts, add characteristics to a contact profile (e.g. contact information, credentials, etc.), and/or to contact a contact. Furthermore, contacts component 318 may be configured to communicate with a contact database, which may store the contacts and related contact information. Such contacts may include one or more caregivers, customer service entities, and/or one or more system managers.

Moreover, user content and services component 302 may include a medical device management component 320, which may be configured to interface and communicate with one or more peripheral or integral medical devices configured to take one or more medical readings of physiological characteristics of a user. Additionally, user content and services component 302 may include a medicine ordering component 322, which may be configured to receive one or more medicine order requests from a user and interface with a pharmacy or other purveyor or medicine to complete the medicine order. In an aspect, medicine ordering component 322 may receive one or more authenticated prescriptions from a caregiver and for a user, may save the authenticated prescriptions, and may block any medicine order request from a user unless medicine ordering component has received an authenticated prescription for the medicine for the requesting user.

In an additional aspect, user content and services component 302 may include a health record component 324, which may be configured to store and/or retrieve a health record associated with a requesting user. Health record component 324 may also communicate with one or more network entities, such as one or more databases, which may store health records associated with one or more users. In addition, health record component 324 may interface with fingerprint scanning component 312 (or other identification security component or device) to ensure that a requesting user is permitted to access a particular health record or health records.

Furthermore, user content and services component 302 may include a calendar component 326, which may be configured to manage a user calendar, which may include one or more appointments (e.g. medical appointments, personal appointments, work appointments), reminders, alerts, or other event notifications. In an aspect, calendar component 326 may further be configured to allow a user to add such an appointment to the calendar, to remove an appointment, reschedule an appointment, and/or edit properties of the appointment, such as a time, location, parties, subject, agenda, or the like.

Additionally, user content and services component 302 may include a television component 328, which may be configured to display one or more television program options or channels to a user via a user interface, receive a television program selection from the user, and present the selected television program to the user (e.g. via display component 334). In an aspect, television component 328 may receive television program content from a remote television server and may display such television program content to the user as streaming video, live video, and/or stored video viewed at a time subsequent to the airtime of the television program content, such as using digital video recorder (DVR) methods.

Moreover, user content and services component 302 may include a telephone component 330, which may be configured to facilitate telephonic communication between a user terminal and one or more remote devices, which may, in some instances, include one or more manager terminals. In some examples, telephone component 330 may utilize VoIP communication technology to facilitate such telephonic communication, though it may alternatively or additionally utilize traditional plain old television service (POTS), or other traditionally wire-line telephonic communication technology. In some examples, telephone component 330 may be configured to initiate and conduct video telephone calls.

In addition, user interaction component 206 may include a user content and services receiving component 332, which may be configured to receive user content and services information and/or data from one or more remote devices (e.g. network entities and/or manager terminals). In some examples, user content and services receiving component 206 may be configured to receive such content wirelessly, and may therefore comprise one or more wireless receivers and/or transceivers. Furthermore, user content and services receiving component 206 may be configured to receive, decode, and/or store in memory such content via a wired connection.

Additionally, user interaction component 206 may include a display component 334, which may be configured to display a user interface, user multimedia or service content, or any other image associated with system 200 of FIG. 2. For example, display component 334 may be configured to receive one or more display instructions or signals from one or more external devices or internal components of user interaction component 206 (e.g. user content and services component 206). In an aspect, display component 334 may be configured to interface with a peripheral or integral display device that may display the content output by display component 334. Such a peripheral display device may comprise a television, flat-screen display, touch screen display, tablet screen, phone screen, monitor, projector, or any other display device or component known in the art. Furthermore, user interaction component 206 may include an input receiving component 336, which may be configured to receive one or more inputs from a user, such as, but not limited to, selections associated with a graphical user interface displayed to a user via display component 336. Such an input receiving component 336 may interface with a peripheral or integral input device, such as, but not limited to, a mouse, touch screen, keyboard, keypad (e.g. smartphone input), microphone, camera, or any other device or component configured to receive input from a user.

In an additional aspect, user interaction component 206 may include a permissions component 336, which may be configured to receive one or more instructions and/or permissions settings from a system manager transmitted, for example, by a remote system manager terminal (e.g., system manager terminal 204 of FIG. 2). Such instructions and/or permissions may control one or more aspects of the user content and services viewed by the user, one or more functions performed by user terminal 202 and/or user interaction component 206, such that the system manager may control the user experience of a user via the remote caregiver terminal. Furthermore, permissions component 338 may receive and set permissions related to one or more services that may be available to one or more users. For example, in some non-limiting examples, permissions component 338 may receive and store instructions that allow or restrict user utilization of services such as, but not limited to, meal ordering and delivery, home and property maintenance, laundry and dry cleaning, and/or any other service that may be utilized by a remote user with permission of a system manager. Additionally, in some non-limiting examples, the system manager issuing permissions received and stored by permissions component 338 may be a remote caregiver. In such examples, manager instruction receiving component 338 may receive a caregiver instruction to prompt a user to take a medical reading associated with a physiological characteristic of the user, such as to step onto a scale or to place a blood pressure monitor on the arm of the user.

Additionally, user interaction component 206 may include a user interface component 340, which may be configured to display and manage a user interface presented to a user with which the user may interact. In some examples, such a user interface may include one or more selection options or menu options corresponding to available aspects of the system 200. Such selection options may each correspond to one of the myriad functional options of the system 200 illustrated in FIG. 1. For example, the user interface presented to the user by user interface component 340 may include, but is not limited to, a telephone call menu, television schedule menu, on-demand film menu, communication menu, video or teleconferencing interface, or medical reading or medical appointment interface.

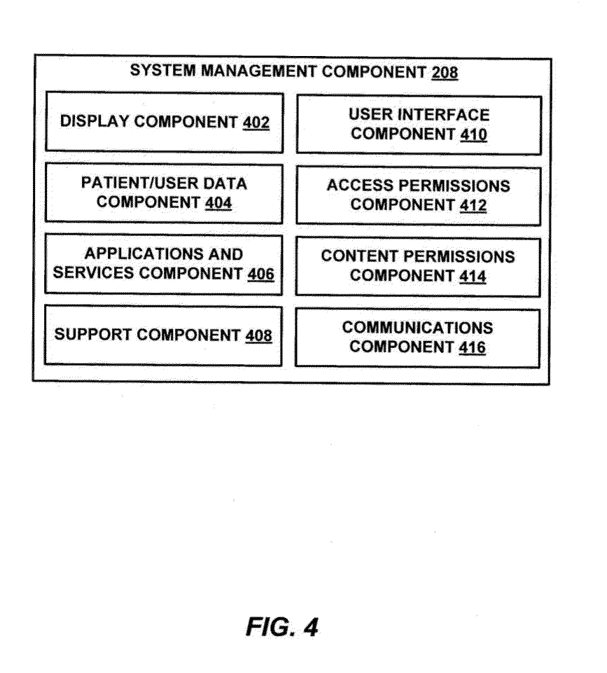

In FIG. 4, a detailed component diagram of system manager management component 208 of system manager terminal 204 of FIG. 2 is presented, which includes several components that may be utilized, alone or in combination, by system manager terminal 204 to provide a user interface for the control the user experience of one or more remote users. For example, manager management component 208 may include a display component 402, which may be configured to display a system manager user interface presenting options for controlling the user experience of one or more remote users. For example, display component 402 may be configured to receive one or more system manager instructions or signals from one or more external input devices. In an aspect, display component 402 may be configured to interface with a peripheral or integral display device that may display the content output by display component 402. Such a peripheral display device may comprise a television, flat-screen display, touch screen display, tablet screen, phone screen, monitor, projector, medical device display, or any other display device or component known in the art.

Furthermore, system manager management component 208 may include a patient/user data component 404, which may be configured to retrieve data, such as profile, medical record, or other relevant data, related to one or more patients or remote users. In an aspect, patient/user data component 404 may communicate with one or more network entities, such as a user or patient profile database or third-party database or server storing patient or user data and may request and receive such data to display to the system manager via display component 402.

Additionally, system manager management component 208 may include an application and services component 406, which may be configured to store a list of available applications and services that are potentially available to one or more users. In an aspect, such applications and/or services may comprise any application, function, or service presented herein, which include content and services, medical content and services, and traditional services including food, property maintenance, and laundry services, for example. Application and services component 406 may store permissions, login, password, contact, address, payment, and/or other information related to such application and service providers for utilization by one or more user terminals. Where such content is deemed accessible to a particular user by a system manager, application and services component 406 may transmit such information to the associated user terminal(s) to allow the user to access such applications and services.

In addition, system manager management component 208 may include a support component 408, which may be configured to manage patient or user support requests. For example, a user or patient may contact the system manager and/or caregiver requesting information, to schedule an appointment, to order a prescription, or indicate a change in medical state, to which support component 408 may indicate to the system manager and/or caregiver. Alternatively, support component 408 may interface with one or more customer service entities to provide system support to the system manager.

Additionally, system manager management component 208 may include a user interface component 410, which may be configured to display and manage a user interface presented to a system manager with which the system manager may interact. In some examples, such a user interface may include one or more selection options or menu options corresponding to available aspects of the system 200. Such selection options may each correspond to one or more functional options of the system manager terminal 204 and/or, system 200, generally. For example, the user interface presented to the user by user interface component 340 may include, but is not limited to, a medical appointment menu, user content control menu, patient or contacts menu, communication menu, video or teleconferencing interface, or medical reading or medical appointment interface.

Moreover, system manager management component 208 may include an access permissions component 412, which may be configured to establish, alter, or otherwise set access permissions associated with user content and services corresponding to one or more patients or user terminal users. For example, access permissions component 412 may view and/or modify user or patient access to web accounts, social networks, web services, medical data, and/or any service contemplated by the present disclosure. Furthermore, system manager management component 208 may include a content permissions component 414, which may be configured to establish, alter, or otherwise set content permissions associated with user content and services corresponding to one or more patients or user terminal users. For example, content permissions component 414 may view and/or modify user or patient access to calendar, music, picture, video or other data.

In addition, system manager management component 208 may include a communications component 416, which may be configured to generate, transmit, and/or receive signals to/from one or more network entities and/or one or more user terminals 202. In an aspect, communications component 416 may comprise a transmitter, receiver, transceiver, and/or related memory and circuitry to facilitate such communication.

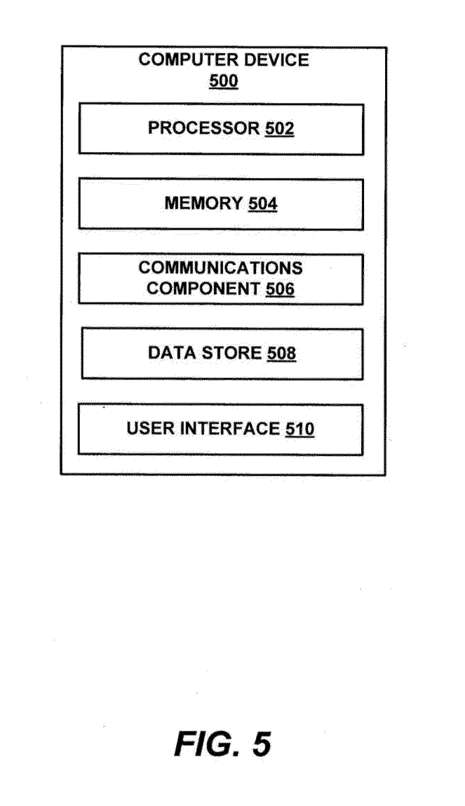

Referring to FIG. 5, in one aspect, any of user terminals 202, system manager terminals 204, and any network entity of network 210 of FIG. 1, or any of the components therein, may be represented by a specially programmed or configured computer device 500. Computer device 500 includes a processor 502 for carrying out processing functions associated with one or more of modules and functions described herein. Processor 502 can include a single or multiple set of processors or multi-core processors. Moreover, processor 502 can be implemented as an integrated processing system and/or a distributed processing system.

Computer device 500 further includes a memory 504, such as for storing data used herein and/or local versions of applications being executed by processor 502. Memory 504 can include any type of memory usable by a computer, such as random access memory (RAM), read only memory (ROM), tapes, magnetic discs, optical discs, volatile memory, non-volatile memory, and any combination thereof.

Further, computer device 500 includes a communications module 506 that provides for establishing and maintaining communications with one or more parties utilizing hardware, software, and services as described herein. Communications module 506 may carry communications between modules on computer device 500, as well as between computer device 500 and external devices, such as devices located across a communications network and/or devices serially or locally connected to computer device 500. For example, communications module 506 may include one or more buses, and may further include transmit chain modules and receive chain modules associated with a transmitter and receiver, respectively, or a transceiver, operable for interfacing with external devices.

Additionally, computer device 500 may further include a data store 508, which can be any suitable combination of hardware and/or software, that provides for mass storage of information, databases, and programs employed in connection with aspects described herein. For example, data store 508 may be a data repository for applications not currently being executed by processor 502.

Computer device 500 may additionally include a user interface module 510 operable to receive inputs from a user of computer device 500, and further operable to generate outputs for presentation to the user. User interface module 510 may include one or more input devices, including but not limited to a keyboard, a number pad, a mouse, a touch-sensitive display, a navigation key, a function key, a microphone, a voice recognition module, any other mechanism capable of receiving an input from a user, or any combination thereof. Further, user interface module 510 may include one or more output devices, including but not limited to a display, a speaker, a haptic feedback mechanism, a printer, any other mechanism capable of presenting an output to a user, or any combination thereof. In an additional aspect, computer device 500 may include or may communicate with one or both of user interaction component 206 and manager management component 208 of FIGS. 1-3.

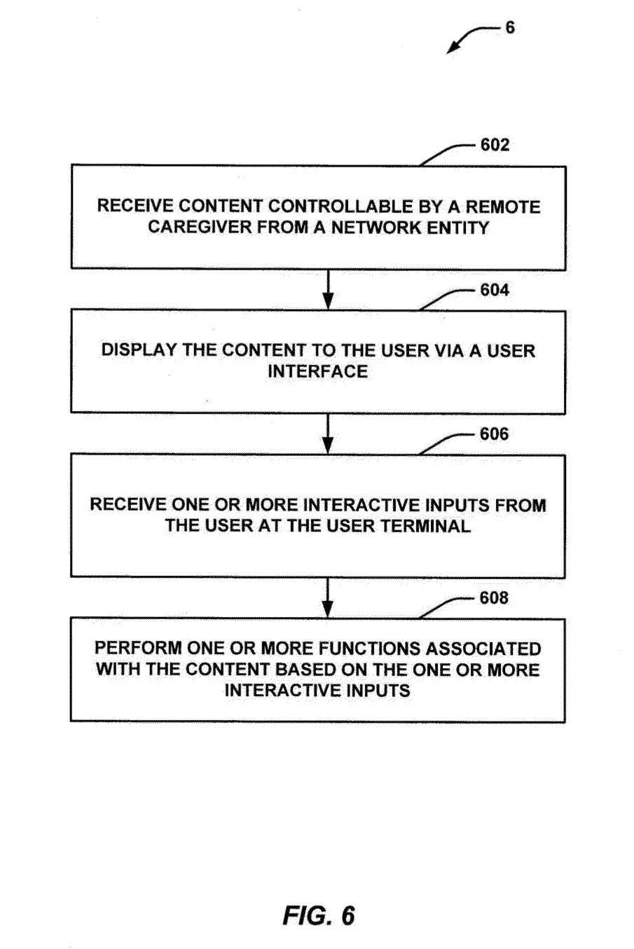

FIG. 6 illustrates an example methodology for providing user interaction with a unified user content and services system at a user terminal. In an aspect, at block 602, the user terminal may receive content from a network entity, wherein at least a portion of the content is controllable by a remote system manager. Furthermore, at block 604, the user terminal may display the content to the user via a user interface, which may be displayed at an integral or peripheral display device. In addition, at block 606, the user terminal may receive one or more interactive inputs from the user at the user terminal, which may correspond to one or more menu or functional options presented via the user interface. Moreover, at block 608, the user terminal may perform one or more functions associated with the content based on the one or more interactive inputs.

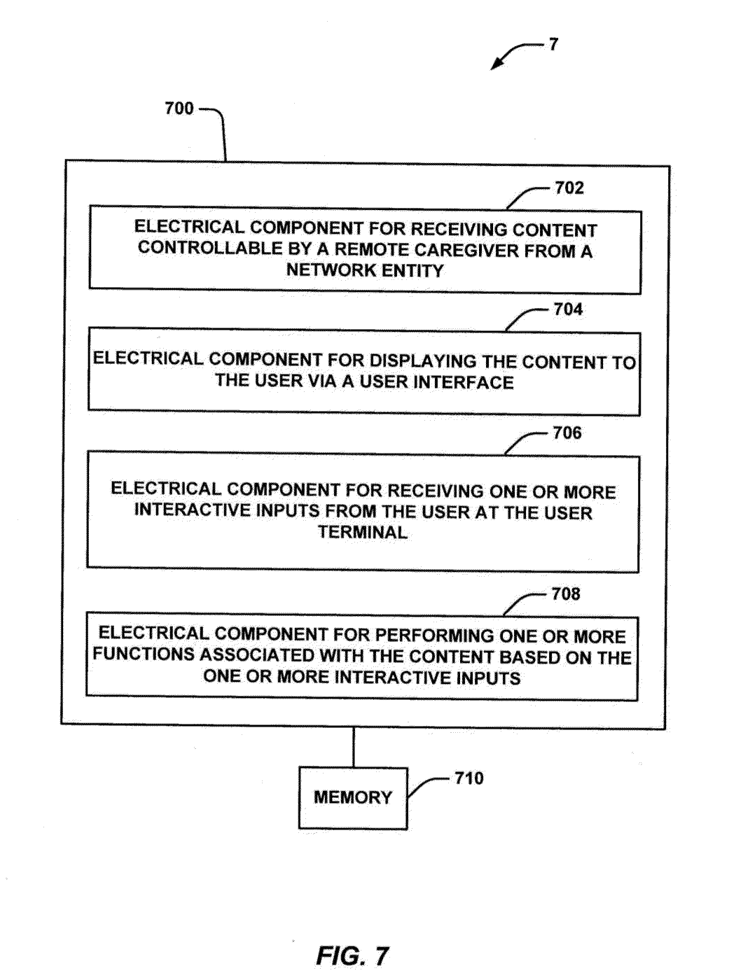

Referring to FIG. 7, an example system 7 is displayed for providing user interaction with a unified user content and services system at a user terminal. For example, system 7 can reside at least partially within one or more computer devices. It is to be appreciated that system 7 is represented as including functional blocks, which can be functional blocks that represent functions implemented by a processor, software, or combination thereof (e.g., firmware). System 7 includes a logical grouping 700 of electrical components that can act in conjunction. For instance, logical grouping 700 can include an electrical component 702 for receiving content controllable by a remote system manager from a network entity. In an aspect, electrical component 702 may comprise user content and services receiving component 332 (FIG. 3). Additionally, logical grouping 700 can include an electrical component 704 for displaying the content to the user via a user interface. In an aspect, electrical component 704 may comprise display component 334 (FIG. 3). Additionally, logical grouping 700 can include an electrical component 706 for receiving one or more interactive inputs from the user at the user terminal. In an aspect, electrical component 706 may comprise input receiving component 336 (FIG. 3). In an additional aspect, logical grouping 700 can include an electrical component 708 for performing one or more functions associated with the content based on the one or more interactive inputs. In an aspect, electrical component 708 may comprise user interaction component 206 (FIGS. 2 and 3).

Additionally, system 7 can include a memory 710 that retains instructions for executing functions associated with the electrical components 702, 704, 706, and 708, stores data used or obtained by the electrical components 702, 704, 706, and 708, etc. While shown as being external to memory 710, it is to be understood that one or more of the electrical components 702, 704, 706, and 708 can exist within memory 710. In one example, electrical components 702, 704, 706, and 708 can comprise at least one processor, or each electrical component 702, 704, 706, and 708 can be a corresponding module of at least one processor. Moreover, in an additional or alternative example, electrical components 702, 704, 706, and 708 can be a computer program product including a computer readable medium, where each electrical component 702, 704, 706, and 708 can be corresponding code.

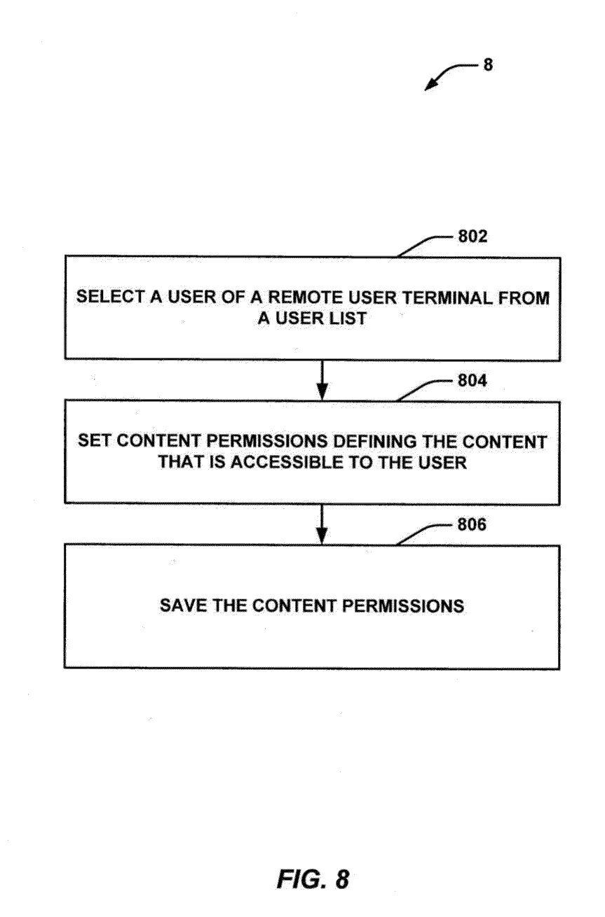

FIG. 8 illustrates an example methodology for controlling user access and user content and services to one or more users associated with a unified user content and services system at a manager terminal. In an aspect, at block 802, the manager terminal may select a user of a remote user terminal from a user list. Furthermore, at block 804, the manager terminal may set content permissions defining the content that is accessible to the user, wherein the content comprises medical content. In addition, at block 806, the manager terminal may save the content permissions, for example, at a remote network entity, such as a database.

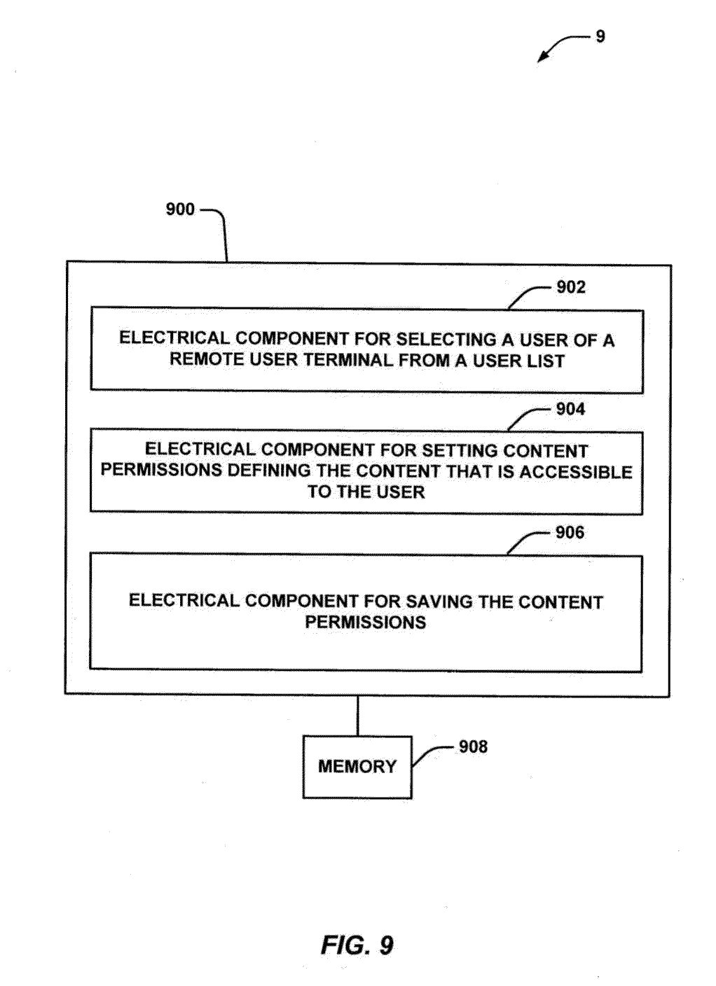

Referring to FIG. 9, an example system 9 is displayed for controlling user access and user content and services to one or more users associated with a unified user content and services system at a manager terminal. For example, system 9 can reside at least partially within one or more computer devices. It is to be appreciated that system 9 is represented as including functional blocks, which can be functional blocks that represent functions implemented by a processor, software, or combination thereof (e.g., firmware). System 9 includes a logical grouping 900 of electrical components that can act in conjunction. For instance, logical grouping 900 can include an electrical component 902 for selecting a user of a remote user terminal from a user list. In an aspect, electrical component 902 may comprise patient/user data component 404 (FIG. 4). Additionally, logical grouping 900 can include an electrical component 904 for setting content permissions defining the content that is accessible to the user. In an aspect, electrical component 904 may comprise one or both of access permissions component 412 and content permissions component 414 (FIG. 4). Additionally, logical grouping 900 can include an electrical component 906 saving the content permissions. In an aspect, electrical component 906 may comprise one or both of access permissions component 412 and content permissions component 414 (FIG. 4).

Additionally, system 9 can include a memory 908 that retains instructions for executing functions associated with the electrical components 902, 904, and 906, stores data used or obtained by the electrical components 902, 904, and 906, etc. While shown as being external to memory 908, it is to be understood that one or more of the electrical components 902, 904, and 906 can exist within memory 908. In one example, electrical components 902, 904, and 906 can comprise at least one processor, or each electrical component 902, 904, and 906 can be a corresponding module of at least one processor. Moreover, in an additional or alternative example, electrical components 902, 904, and 906 can be a computer program product including a computer readable medium, where each electrical component 902, 904, and 906 can be corresponding code.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

In some embodiments, the interactive terminal described herein combines a multipurpose digital screen with a highly simplified remote that creates automated use of pre-provisioned access to a wide range of services and connections. This structure creates a level of integration and simplified access that is a tradeoff between the benefits of automated access and user control or flexibility. Simplified access is achieved by taking away user control and flexibility, and automating access to the connections and services a user or their approved network wants to have available on their system. The system also includes the ability of the user or a third party to provide a schedule that provisions and changes the available automated connections and services for the benefit of the primary user and their approved network that benefits from automated access to the user.

Services accessed through the screen and remote include multichannel video programming distributor (MVPD) TV, access to all basic broadband connections, information and services, access to internet supported video conference calls, access to both Voice-over-Internet Protocol (VoIP) and cell phone voice calls, internet hosted video calls, and secure access to a wide range of medical service information and connections. The system structure enables cost effective delivery of medical services on a secure virtual private network (VPN) platform to many parties that would otherwise not be able to have access to them.

The remote provides access to all services via a simple 12 button structure. Buttons are also larger and arranged with enough spacing to facilitate easy use as compared to today's typical 30+ button remotes with very tight spacing and small print text to identify their use. The remote is also based on a WiFi or other active wireless connection that eliminates the need for the remote to have a “line-of-sight visual” relationship to the screen to be able to control the system as is required for infrared TV remotes. In other words, this remote not only controls the system when it is not pointed at the screen, but also is capable of controlling the system from a different room.

The telephone may also be a highly simplified handset. In some embodiments, the telephone is a hybrid phone that combines a Voice over Internet Protocol (VoIP) structure in the residence with a cell phone connection whenever the phone is out-of-range of the VoIP connection. This telephone is expected to provide the highest level of connection and security possible for users. If the VoIP connection in the residence fails for any reason, the phone automatically seeks a cell phone connection as a backup. Global positioning system (GPS) plus cell system location technology may be used to provide the highest level of location information. Emergency calls can include interaction with a customer service operator who has approved access to personal customer medical and other information to assist any response team.

The simplified user interface may be coupled with the ability of a customer service agent to remotely control and provision the user's system. In some cases, the customer service agent is available to the user at any time of day. Simplicity for the user is mirrored by increased complexity at the customer service operations.

With today's broadband networks and modifications to existing remote control software, the operator can remotely control all of the services available on the interactive system. Simplified control screens are developed for each element of the system that the customer service operator manages and changes on behalf of a user. This primary operator may be backed up by an advanced technical support group that can remotely control the system dedicated to a user. The operator may also act as a concierge with the ability to direct a user to different broadband sourced information or services that are desired by the user or the user's personal network.

The managed network provided additional operating benefits to hospitals and housing or service management organizations. Hospitals can replace their cable TV networks with broadband video networks that are capable of not only delivering traditional TV, but also support live video connections from any point on the network to any other point. When a patient is admitted to the hospital or other care facility, the unit in their room can be provisioned to support all the personal network connections and access to entertainment that they desire. Nurses, doctors and other hospital staff may also have video, speaker phone, or text access to each patient. Since the connections are pre-programmed, hospital staff may also have automated access to family or external caregivers as may be needed for consultation or the ability to provide care updates. Education and training may be enhanced by providing patient access to a Web based library of accessible video material that can be ordered for the patient to use in the hospital or as follow up after discharge. Communications on the internal broadband network can also automatically update the Electronic Medical Record system and be recorded for billing or liability issues.

Organizations responsible for senior or disabled housing can have similar automated secure access to their managed residences in multi-dwelling units or to remote single housing locations. The secure broadband connections also provide opportunities for cost effective security and monitoring that can improve service and lower cost.

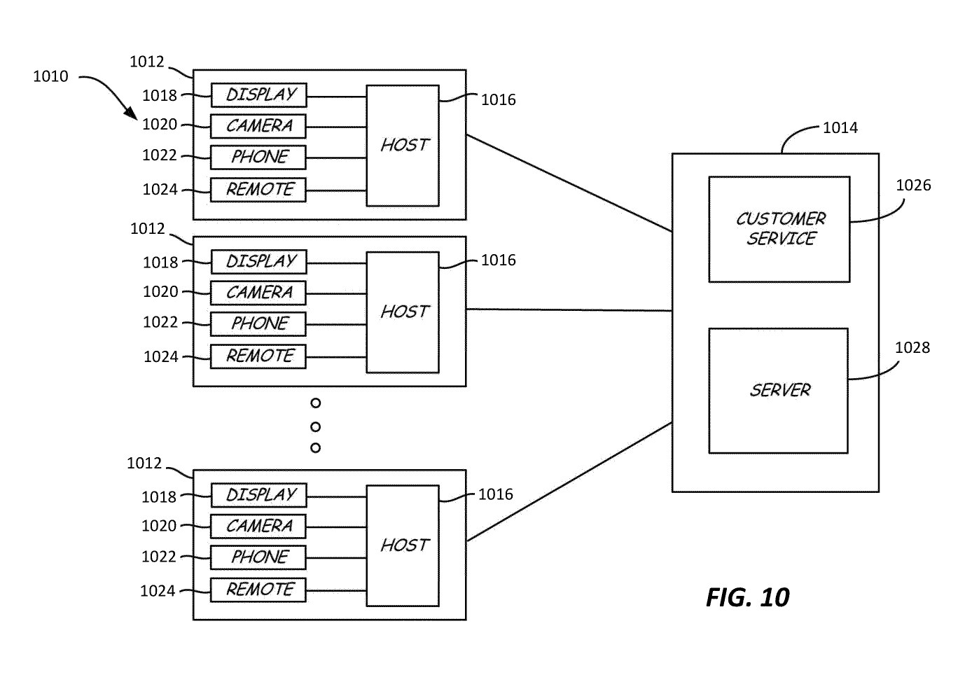

FIG. 10 is a block diagram of a system 1010 for communication and media distribution to one or more interactive terminals 1012. The interactive terminals 1012 communicate with a centralized systems portion 1014. While three interactive terminals 1012 are shown, the system 1010 may include any number of interactive terminals 1012 that communicate with the centralized systems portion 1014. The interactive terminals 1012 each include a host 1016, a display 1018, a camera 1020, a telephone 1022, and a remote control 1024. In some embodiments, the host 1016 communicates with and/or controls operation of the display 1018, camera 1020, and telephone 1022. The host 1016 may also coordinate communication of the interactive terminal 1012 with the centralized systems portion 1014. The remote control 1024 may be used to communicate with the host 1016 to interact with and control certain operations of the host 1016. Signals between the interactive terminals 1012 and the centralized systems portion 1014 may be routed through a central communications port, or the interactive terminals 1012 may each communicate with the centralized systems portion 1014 directly.

The centralized systems portion 1014 includes a customer service resource 1026 and a server 1028. The customer service resource 1026 and server 1028 may be provided at the same or different locations. When in different locations, the customer service resource 1026 may be located at a customer center, and the server 1028 may be located at the same facility as the interactive terminals or another remote location, for example. As will be described in more detail herein, the customer service resource 1026 provides assistance to users of the interactive terminals 1012 and works with the users and/or third parties to control menuing and provisioned content and service options for the users. The server 1028 may store information and content that is provided to the interactive terminals 1012.

The interactive terminals 1012 may be provided in a private home, an eldercare facility, the residence of a disabled person, or the patient's room in a healthcare or eldercare facility, for example. The interactive terminal 1012 may be connected to a locally or remotely located server 1028 or centralized systems portion 1014 that provides and controls access to the provisioned services and information. In addition to the components shown, the interactive terminal 1012 may include local media players and devices, such as a video player (e.g., DVD or Blu-Ray player) and/or a personal computer, that are provided in the host 1016 and accessible through the menuing system on interactive terminal 1012. Alternatively, some or all media players and devices may be located at and accessed through the centralized systems portion 1014.

The host 1016 may be a box-like structure that provides access to and control of multimedia and data services for the interactive terminal 1012. Alternatively, the host 1016 may be a multimedia personal computer. The host 1016 communicates with the centralized systems portion 1014 to provide the user with provisioned services and information, and to provide access to the customer service resource 1026. The host 1016 includes connections or devices to provide connectivity with each of the elements (display 1018, camera 1020, telephone 1022, remote control 1024) of the interactive terminal 1012 and other external devices (e.g., a computer, portable hard drive, video camera, still camera, etc.). For example, the host 1016 may include audio/video connections (e.g., HDMI, DVI, component video, coaxial, etc.), data connections (e.g., Ethernet, USB, FireWire), and/or wireless connections (e.g., radio frequency, infrared) The host 1016 may further include an integrated local hard drive to store media (e.g., photos, video, etc.) for the user.

The display 1018 may be a television that receives audio and video signals through the host 1016. For example, the host 1016 may receive signals from a source of cable or satellite television, and provide the signals to the display 1018 as an output. In an alternative embodiment, the display 1018 is a computer monitor. In some embodiments, the display 1018 is configured with any 720p or greater resolution. The display 1018 may be a digital television of any size that has inputs for both a PC and TV signal using application software that takes control of the primary user functions and related displays. This display 1018 can use a fully integrated multimedia disc player or also be configured with a separate multimedia disc player that can be controlled with the remote control 1024 as configured.

The camera 1020 may also have video and still image capturing capabilities. In some embodiments, the camera 1020 is integrated with the display 1018. This provides an interface for video conferencing, for example, wherein the user can communicate via a broadband connection with another party.

The camera 1020 used by the video call service is also available for security or other observation of the residence. The camera 1020 may have a fixed wide view capability with auto focus. The camera 1020 may be positioned to capture video of the user's bed and surrounding areas in the user's in-patient or in-home room, for example. More sophisticated cameras 1020 may also be available that pan and scan the user's room. The system may also be capable of hosting additional remote cameras that may be desired for security, user (e.g., patient) monitoring, or resident management. These cameras may also be capable of remote activation.

Normal control for the camera 1020 to be available and active or turned off is provided to the primary user of the interactive terminal 1012. The camera 1020 may also be capable of remote activation where remote activation has been legally approved by the user under certain circumstances. For example, the camera 1020 may be remotely activated by a nurse to check on the status of the user, if the user has approved such monitoring. Activation may also be approved for customer service or for members of the user's network that communicate with or provide service to the user.

The audio and video produced by the camera 1020 may be recorded if desired for future reference.

The telephone 1022 associated with the interactive terminal 1012 may be a hybrid phone that combines the use of VoIP connections in the residence location and cell phone connections whenever the hand set is out of range of the residential base station. This telephone 1022 may include a simplified keypad similar to the twelve button remote control 1024. The telephone 1022 may provide direct access to a customer service operator at the customer service resource 1026.

When any phone call is received by the system 1010, a record of that call may be stored in the system 1010, and a caller identification message is also sent to the display 1018 so the user can determine if he or she wants to answer the call or not. The same preprogrammed speaker phone connections that are available through the screen speaker phone system (described herein with regard to FIG. 17) may automatically be available as auto dial numbers on the telephone 1022. In addition, recorded voicemail messages available through display 1018 (described herein with regard to FIG. 15) may also be retrieved using the telephone 1022.

Since the telephone 1022 is both cell phone and VoIP capable, the cell phone connection becomes the emergency backup link if the VoIP connection fails for any reason. The telephone 1022 may also host GPS location technology that relays location to the customer service operations. The combination of GPS technology, VoIP technology that verifies if someone is home, and cellular location technology creates an environment for emergency response that provides a high level of location information. Broadband technology also provides remote access for a customer service operator to control any settings on the telephone 1022.

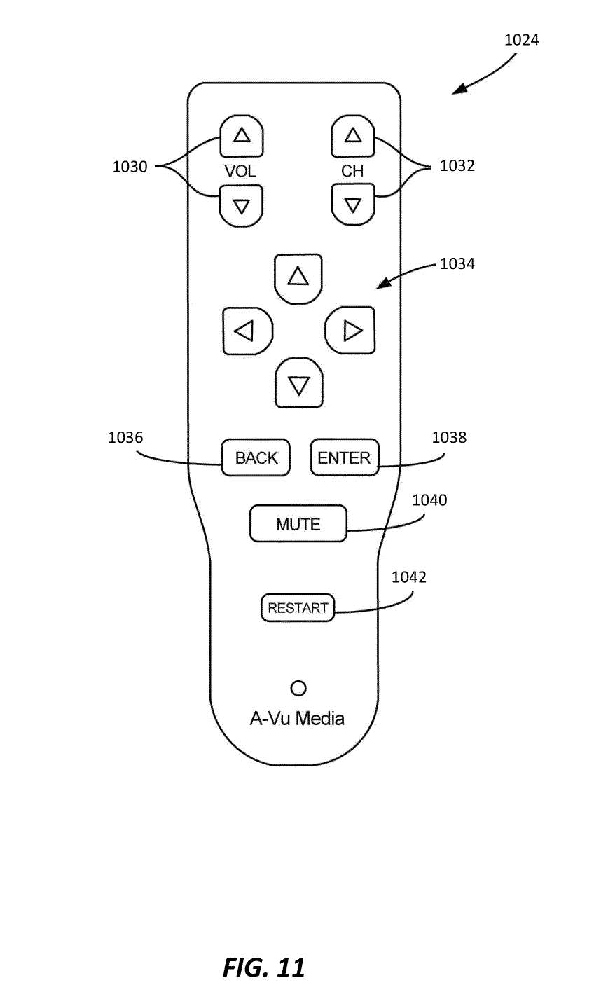

The remote control 1024 provides system navigation and service activation for all remotely managed services in the interactive terminal 1012. The remote control 1024 may be based on a WiFi or other active wireless connection that eliminates the need for the remote to have a “line-of-sight visual” relationship to the screen to be able to control the system as is required for infrared TV remotes. In other words, the remote control 1024 not only controls the interactive terminal 1012 when it is not pointed at the screen, but also is capable of controlling the interactive terminal 1012 from a different room. An exemplary remote control 1024 is shown and described below with respect to FIG. 11.

At the centralized systems portion 1014, the customer service resource 1026 may be a computer or computer network operated by one or more customer service operators. The customer service resource 1026 may be accessed by the interactive terminals 1012 via a local area network or via the internet, for example. The customer service resource 1026 may be located locally at the facility in which the interactive terminals 1012 are located, or the customer service resource 1026 may be located remotely in a facility dedicated to providing customer services for users of the interactive terminals 1012. The customer service resource 1026 may include voice and video communication capabilities, allowing the customer service operator to communicate with the users of the interactive terminals 1012, healthcare professionals, and other third parties.

The customer service operators and/or authorized third parties (e.g., family members) may communicate with the interactive terminals 1012 to provide to control the content and services provisioned at each of the interactive terminals 1012. This schedule of provisioned content and services may be selected by the user of the interactive terminals 1012, or by a third party who has been given authority to select the content and services available to the user. For example, the customer service operators may have automated access to all users with the ability, under predefined emergency or other conditions approved by the user, to override and activate the camera 1020 that has been turned off by a user.

The customer service operator and/or authorized third parties may also have remote control of some or all services offered on the system. These remotely controlled services include but are not limited to: (1) activating or deactivating TV channels, (2) activating new video services purchased by the user, (3) downloading new video content for future viewing, (4) deleting video content from storage (e.g., in host 1016 or server 1028), (5) downloading new music content for storage, (6) deleting music content from storage, (7) remotely controlling the structure of storage for the music by creating music categories and moving stored music to new locations, (8) activating or deleting music channels from the MVPD provider or the Internet, (9) downloading new photos for storage, (10) deleting unwanted photos, (11) creating new photo albums and reorganize the storage of photos, (12) approving or deleting any third party's access to the user for the ability to send any form of information or to automatically connect via a video call, (13) e-mailing or otherwise electronically sending any software to approved user network members that need compatible software for the automated connections to the interactive terminal 1012, (14) deleting any unwanted text or voice messages, (15) adding or deleting access to specific websites, (16) adding or deleting calendar or other message reminders that are provided on the display at appropriate times, and/or (17) adding or deleting any individuals, medical providers, community services, or commercial service providers for automated text, speaker phone or video phone connections.

The customer service operator may have access to the personal network of the user and user approved medical information that can be a resource in the event of an emergency or other condition where help from these third party resources would be of value. The customer service may also be provisioned as the first point of contact for an emergency call or it can be an automated secondary resource for any standard emergency (e.g., 911) call to other external emergency service providers.

The operator may also have access to a broad range of services and/or information resources that can be offered to the user. In this regard the customer service operator acts as a concierge for the user to help them find access to relevant information or services that they may not realize are available to meet their identified needs or interests. The operating system for the operator may provide simplified screens that automatically populate with the current structure and information for a user while also providing access to change the settings, content, or other controllable conditions that are present.

The operator may have non-stop access to a technical support team that has remote PC administrator control of the user's system using a secure VPN connection. The structure for the operator may provide for remote work locations that would allow an operator to work from home or a remote location with part time or full-time status.

The server 1028 may be located in the same facility in which the interactive terminals 1012 are located, or the server 1028 may be located remotely. The server 1028 may be accessed by the interactive terminals 1012 via a local area network or via the internet, for example. The server 1028 may store content available for access by all interactive terminals 1012, such as movies, music, and games. The server 1028 may also store data, information, and media specific to each of the users of the interactive terminals 1012. For example, the server 1028 may store a user's preferred or stored music or movie selection, photos and videos received by the user (e.g., via email), phone messages for the user, and/or messages and information from a doctor or other healthcare provider specific to the user. The server 1028 may also store general menuing and user interface data and information for the interactive terminals 1012.

FIG. 11 shows an embodiment of the remote control 1024 suitable for use with the interactive terminals 1012. The remote control 1024 includes two volume (VOL) buttons 1030 control the speaker volume for all hosted services that produce any audio through the speakers of the primary system. The remote control 1024 also includes two channel (CH) buttons 1032 that provide selection control of television channels. A single press of either button 1032 moves the system up or down one channel. A longer hold of either button 1032 creates a fast up or fast down changing of channel selection.

The four arrow buttons 1034 in the middle of the remote are navigation buttons to move a cursor or the selection of an option presented on the screen to the active mode for use. If the active item on the screen is at the very top, bottom, left or right position on the screen, pushing the button to move it farther up, down, left or right produces no change on the screen.

The back button 1036 moves the system back to the previous screen and related services that was presented for the user to have access to. If the user is at the default start screen, pushing the back button 1036 does not provide a response.

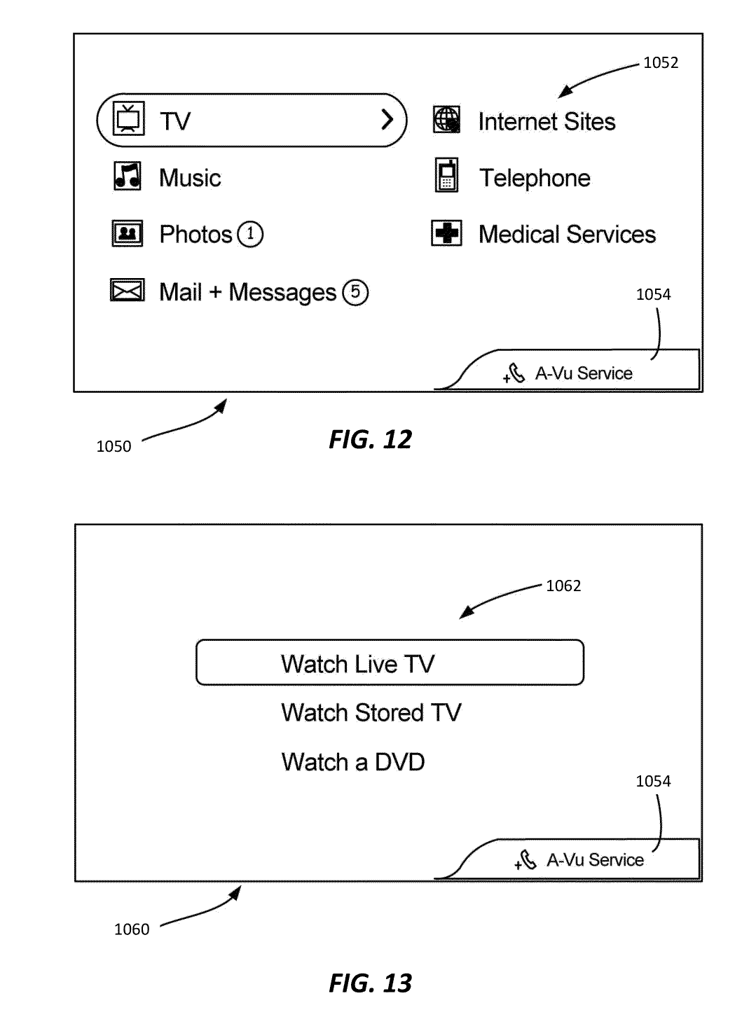

The enter button 1038 activates the highlighted item as displayed on the current screen. For example, in FIG. 12 (discussed in more detail below), the TV option is highlighted on the screen, so pressing the enter button 1038 would initiate the TV services provided through the interactive terminal 1012.

The mute button 1040 is active when the system has active audio. In some embodiments, pressing the mute button 1040 the first time cuts the audio volume down to 50% of its current setting. Pressing the mute button 1040 twice takes the system to full audio mute. Pressing the mute button 1040 a third time restores the audio volume setting to its setting prior to pressing the mute button 1040 the first time.

The restart button 1042 can be pressed at any time to cause the system to return to the default start screen (FIG. 12) for access to services. In some embodiments, if the restart button 1042 is pushed three consecutive times in a short time period (e.g., less than three seconds), or if the restart button 1042 is held down for a longer period of time (e.g., 5-10 seconds or more), it also creates an automatic speaker phone call to the customer service center.

FIG. 12 is a screen view of a top level or primary screen 1050 that provides access to provisioned services available on an interactive terminal 1012 via a graphical user interface on the display 1018. The screen view 1050 of FIG. 12 shows one possible top level menu 1052 of the provisioned primary services that may be offered to individual users. It will be appreciated that other menu configurations with other menu options are also possible. The screen view 1050 presents a configuration of seven separate services plus direct access to customer service button 1054 in the lower right corner. Access to customer service may be provided on all primary screen configurations.

The seven primary services presented in the menu 1052 in FIG. 12 include TV, Music, Photos, Mail and Messages, Internet Sites, Telephone, and Medical Services. A common characteristic is that each of these services can be selected and accessed/activated using the remote control 1024. If a service is highlighted on the menu 1052, as the TV service on FIG. 12, the enter button 1038 may be pressed to provide automatic access to that service. In some embodiments, selecting a primary service on the menu 1052 links to a sublevel menu associated with the selected primary service. At least some of the options on the sublevel menu link directly to media content or a communication service.

The software associated with the interactive terminal 1012 enables the addition or subtraction of specific services from the menu 1052 and is capable of reordering the sequence of the services presented in the menu 1052.

While not shown in FIG. 12, a portion or portions (e.g., windows) on the screen may also be provisioned for real-time temporary messages. For example, the bottom portion of the screen (e.g., next to the direct access customer service button 1054) can be reserved for temporary banner types of messages that the system can be configured to display providing further temporary information for the end user.

FIG. 13 is a screen view of a TV service screen 1060 including a menu 1062 that is provided on the interactive terminal 1012 for selecting from available television services on the system. This service provides access to live television programming via the “Watch Live TV” option or stored television programming and other stored media via the “Watch Stored TV” option. The screen 1060 also provides access to play a DVD (located locally or remotely from the interactive terminal 1012) via the “Watch a DVD” option.

Selecting the Watch Live TV option on the menu 1062 provides access a standard line up of live TV channels or on-demand channels offered by an MVPD Video provider (e.g., cable, satellite, or internet). After the user has selected this service mode the interactive terminal 1012 functions with standard TV channel selection and use. The package of TV channels and value added services are selectable by the end user. The system is also capable of deleting unwanted channels from the line-up of options that are seen by the end user.

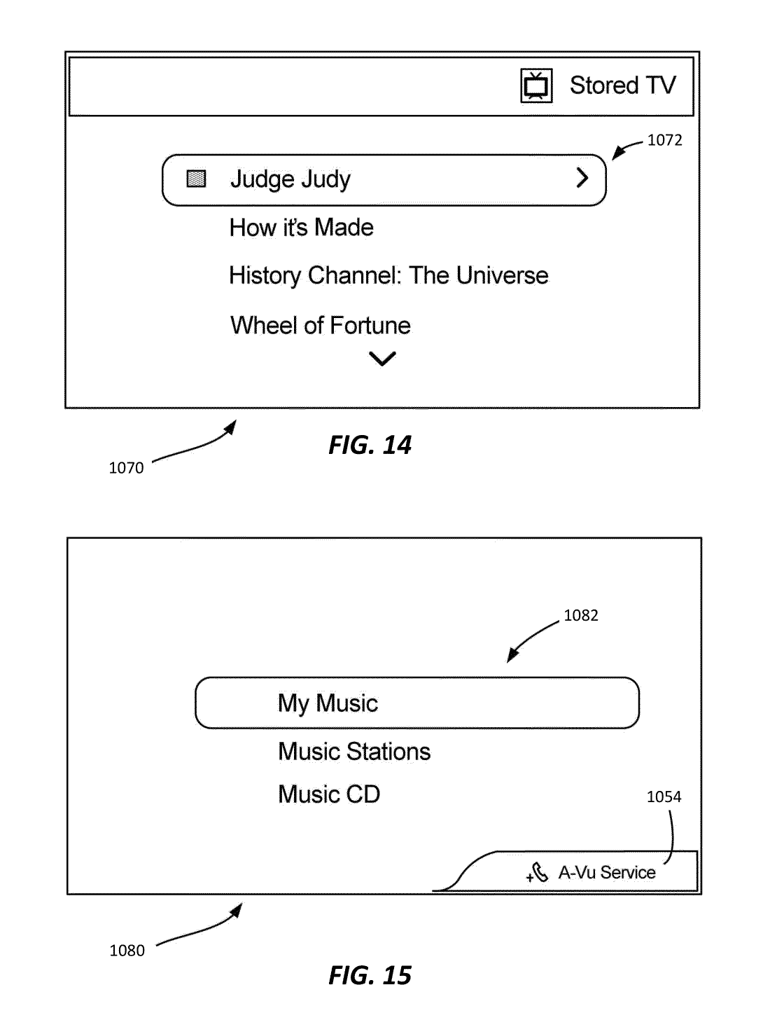

Selecting the Watch Stored TV option on the menu 1062 provides access to a list of stored (i.e., recorded) video that is available to watch at any time the user chooses. A list of available titles, illustrated in stored TV screen 1070 in FIG. 14, shows a menu 1072 of some or all video material that is available. The screen 1070 may include an intermediate menu that lists the categories of stored video available for viewing. In any case, the menu 1072 includes video material that has been provisioned for recording and storage by the customer service center associated with the interactive terminal 1012 that is recorded on a regular basis or as a non-recurring recording.

The video available on the menu 1072 is electronically stored at either a local hard drive dedicated to the user's location (e.g., on host 1016) or at the server 1028 when appropriate. Stored video that is conditioned for automatic access can also be movies or other material that is downloaded from the internet by request of the user or sent to the user by approved members of the user's network. For example, a family member may order a movie from a video rental business or some other provider that automatically shows up in the list for viewing. Other third parties with permission may also send other video based material for education, exercise, or other purposes that also shows up for viewing on this primary list.

The selection of any listed video content on the menu 1072 results in the automatic play of this content on the host 1016. A screen may be displayed at the end of the video asking whether the user wants to store or delete the video content that has just been seen. A customer service operator has the ability to delete material as may be requested.

Selecting the Watch a DVD option on the menu 1062 of screen 1060 in FIG. 13 automatically activates the DVD player, which is also controlled by the remote control 1024. If no DVD is detected in the player the user may be prompted to insert one and then the system moves forward to onscreen controls to start, pause, or stop the DVD. At the conclusion of the DVD, the DVD may be automatically ejected for the user to remove and store.

FIG. 15 illustrates the primary music service screen 1080 including a menu 1082, which appears when the Music option is selected from the menu 1052 in FIG. 12. Activating this service provides access to three basic sources for music that plays through speakers associated with the interactive terminal 1012 or speakers provided in the user's room. Selecting the “My Music” option provides an on screen listing of all electronically stored music. Highlighting and activating any specific title causes the media associated with the title to play on the system. The title may represent stored music or any other stored audio material, such as audio books, or other audio only material of any nature. Music that is available through the interactive terminal 1012 could be entered locally on the system (i.e., on host 1016), stored on server 1028, or downloaded electronically from the internet. Either the customer service associated with the interactive terminal 1012 or approved members of the user's network may have the ability to download music for storage.

Selecting the “Music Stations” option on menu 1082 gives the user direct access to music stations provided by the MVPD or available over the internet. Internet music sites available through this option may be provisioned by customer service at the request of the user.

The system may also play a compact disc (CD) that has been inserted into a multimedia disc player associated with the host 1016. If the user selects the “Music CD” option on the menu 1082 and a CD is available on the player, the CD begins to play automatically. If the Music CD option is selected but a CD is not loaded into the player, the user may be prompted to insert a disc. The CD player is controlled through on-screen menus and interfaces navigated with the remote control 1024. When the CD has finished playing, the host 1016 may prompt the user whether he or she wants an electronic copy of the music on the CD to be stored on the system for future play (e.g., via the My Music option on screen 1080).

The photos screen 1090 shown in FIG. 14, which appears when the “Photos” option is selected from the menu 1052 in FIG. 12, provides automatic access to photos stored on the system. The photos may be stored locally on the host 1016, or remotely at a centralized systems portion 1014/server 1028 as described above. In any case, electronic photo albums may be configured by customer service. Digital photos may be provided from any type of media. For example, the multimedia disc player may have the capability to detect and upload photos to a new album from a CD, and the system may include a USB port for the uploading of stored material. Printed photos may be scanned or otherwise populated into the electronic photo albums with material. The user's approved network may also be given the ability to send photos via e-mail that are automatically accepted and stored for the user's viewing. The photo screen 1090 includes a navigation bar 1092 to scroll through the photos available for viewing.

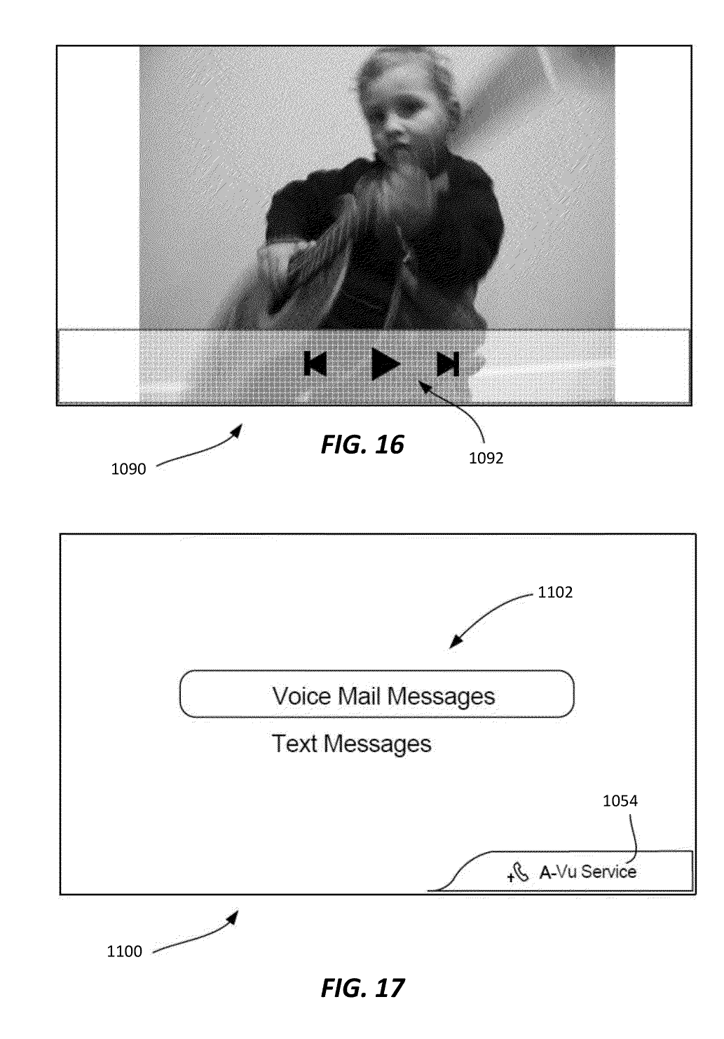

The “1” provided next to the Photos option in FIG. 12 indicates that the interactive terminal 1012 has one new photo that has been received but not viewed. If the condition of “new photos” exists in the system and the user selects the Photos option on menu 1052, the new photos may be automatically presented as the first option in the list of stored photo albums. If the user accesses the Photos section and no new photos are available, the user has access to all of the photo albums and related material available on the system for viewing. In any case, when the user activates this choice, the photo(s) are automatically displayed on the screen for viewing as illustrated in FIG. 16.

To accomplish this level of automation, the approved parties on the user's network may be provided software that is attached to the emails they send to the user's e-mail address that automatically strips the photo from the e-mail and routes it to the appropriate electronic photo album for storage and automated access. The user's software then sends a notation on the primary screen 1030 informing the user of the availability of new photos to view. This automation can also be applied to new photo material that is associated with the user from other sources, such as social networking websites, photo sharing websites, and the like.

The Mail and Messages option on menu 1052 in FIG. 12 provides automated access to voice and text messages from multiple resources including standard voice mail from the phone system, text messages from a mobile phone system, text messages from e-mail, or messages from social networking sites. The “5” next to the Mail and Messages option indicates that there are five new messages that have not been viewed or listened to by the user.

The mail and messages screen 1100 shown in FIG. 17 is displayed when the user selects the Mail and Messages option in menu 1052. This screen 1100 includes a menu 1102 that divides the user's message box into Voice Mail Messages and Text Messages. The number next to each of these options indicates the number of messages of each type waiting for the user to process.

When the Voice Mail Messages option is selected from the menu 1102, the system provides direct access to a list of Voice Mail Messages stored on the system. New or unprocessed messages may be provided at the top of the list. When retrieved, the message is played through the primary speakers of the interactive terminal 1012. At the end of processing any message the user may have the option to store or delete the message.

Text messages are processed in the same way that voice mail messages are processed when the Text Messages option is selected from the menu 1102. The user may have the option of different size and styles of font that can be provisioned by customer service. Text messages that do not fit on one screen may be accessed by using the two middle navigation buttons 1034 on the remote control 1024 to scroll up or down on the text.

Text and/or voice mail messages may be date and time stamped, and may also be identified by sender. When a new message is received, the user may have the option to have a temporary message appear on the screen providing the sender's name and type of message received. This message can appear regardless of what other functions or services are active on the screen. Received messages may be processed as either voice or text messages or both. Voice to text and text to voice software options may be offered as part of the service package.

Voice and/or text messages may be responded to via speaker phone response. If the recorded response is in reply to a text message, it may be converted to a text response. In some embodiments, if the user is responding to a text message, the text of the converted voice response may be displayed on the screen for verification before sending.

Higher functioning users may be given the option of a simplified wireless keyboard that can support text responses to messages. A text message created in this manner can also be converted to a voice message response.

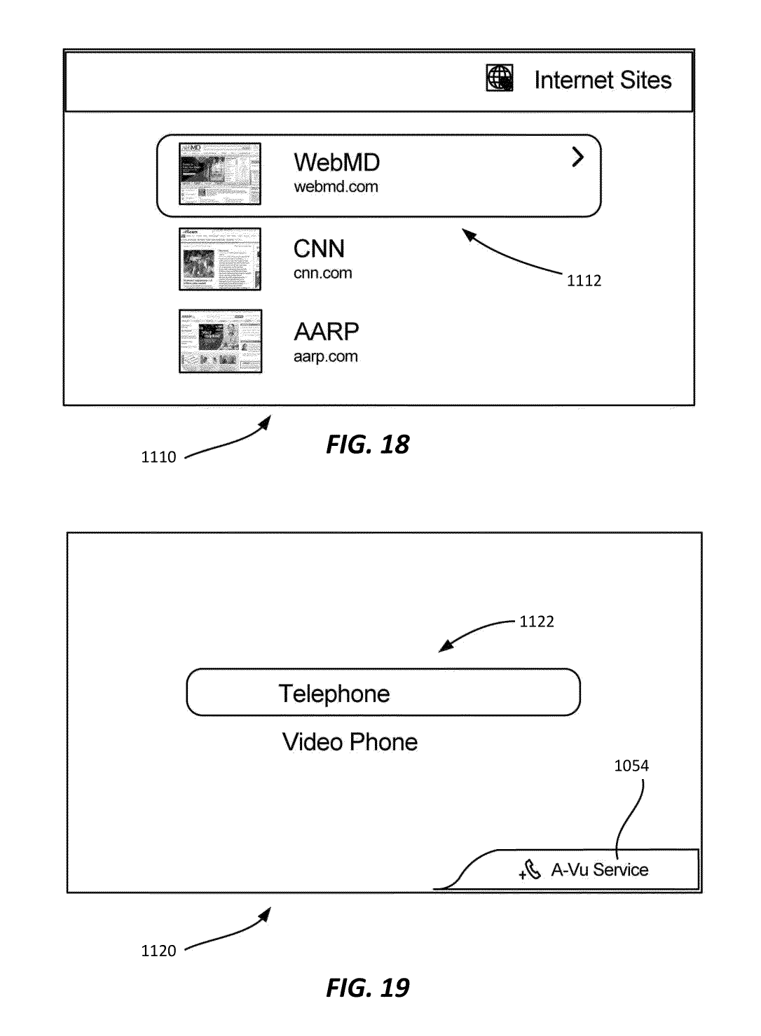

The Internet Sites option on menu 1052 in FIG. 12 provides direct access to internet sites that have been approved and provisioned for the user. When a user selects the Internet Sites option, the screen 1110 displays a menu 1112 including a list of approved Internet sites for the user, as shown in FIG. 18. User access to the internet or internet based resources access to the user may be controlled by customer service. In some embodiments, there is no access to the user's address unless there is specific permission to have access and the user is not permitted to do open access Internet searches. This secure controlled structure is sometimes referred to as a “Walled Garden” structure.

The Telephone service screen 1120, which includes a menu 1122 and is displayed when the Telephone option is selected on menu 1052 in FIG. 12, provides access to either automated speaker phone or video phone connections, as shown in FIG. 19. Speaker phone connections may be preprogrammed for automated access. Description of the available connection may be done with text and numbers shown on the display 1018. A photo associated with the party talking to the user may also be displayed on the display 1018. Speaker phone service may be provided through system speakers and a microphone associated with the interactive terminal 1012, or it may be provided through the telephone 1022.

If a user accepts a speaker phone call while other services are active, the audio for the other service may be automatically muted. The system may be configured to pause the other service for the duration of the speaker phone call. For example, the digital video recording functions of the system may be used to pause the programming when viewing live or broadcast video.

The video phone option may also have pre-programmed connections that have been provisioned by customer service. That is, the system may be programmed to accept video phone calls from only approved parties. The listing of provisioned video phone calls may also include both text and photos of the connection that is available. If a user accepts a video phone call while other services are playing, the audio for the other service is automatically muted and the screen image shifts to the video call for the full screen or display the video conference call in a window on the screen in front of the pre-existing screen material. The system may be configured to pause the other service for the duration of the video phone call. For example, the digital video recording functions of the system may be used to pause the programming when viewing live or broadcast video.

If the user or an approved third party tries to initiate a video call and the connection is not available, the system defaults to processing a voice mail message for receipt by either the user or a member of the user's approved video call network.

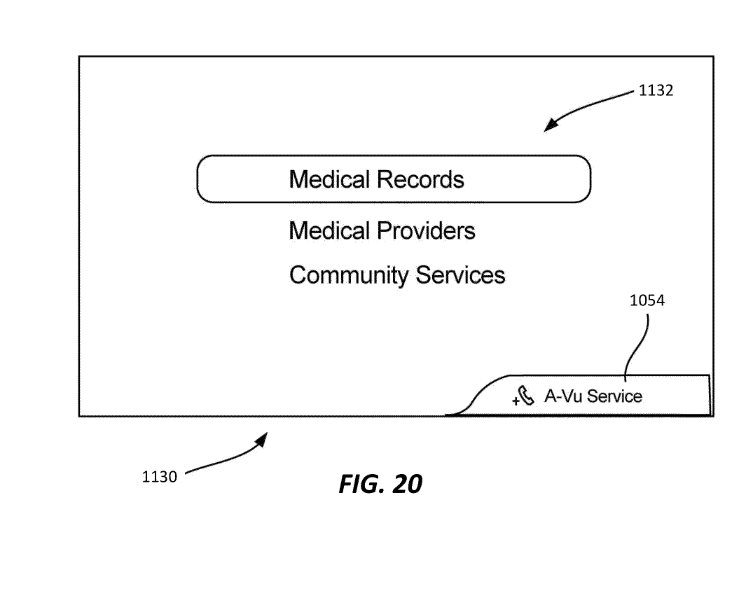

The Medical Services screen 1130 in FIG. 20 is provided on the display 1018 when the Medical Services option is selected from menu 1052 in FIG. 10. The Medical Services screen 1130, which includes a menu 1132 functions as a portal for the user to have automated access to a variety of medical services, community services, or other professional caregiver resources. Connections to the services, organizations, or individuals through the Medical Services screen 1130 can be via text, speaker phone, or video phone. Any connection through this set of accessible services is provided with a VPN connection so that these services are secure and compliant to any HIPAA or other medical service requirements. Any connection through this service portal is also available for electronic recording for liability or billing requirements.

Websites and the communication infrastructure for the connected services may be upgraded to be compliant with the platform associated with the interactive terminal 1012 and the remote control 1024 that may be used by the user to access information, people, or services offered by the third party they are connecting with. Connected third parties may also be given approved access to connect with the user for a live connection or to send information that they want the user to have access to. Access to the user's medical records may be PHR and EMR agnostic.

The system may host a pre-conditioned wireless connection (WiFi and other) that can be used to host medical and non-medical devices that caretakers or health care providers wish to activate in the residence of the user. Medical monitoring or other devices are approved for compatibility so that they automatically register and start to function when they are brought into range of the system they are registered to operate on.

The system has a controlled security structure that complies with HIPAA Security Rules. For example, a fingerprint scanner may be used as the final security confirmation before granting access to controlled medical information.

Healthcare Facility Applications

The system described has value and operating characteristics when implemented as an internal system for the operations of a hospital or other healthcare facility. The system in the hospital or clinic operations can be accomplished with a central server model or with the use of the same distributed model used for private residences.

Patients may be interviewed at admission to determine what outside connections they would want to have available to them from their rooms. This connection and desired entertainment information may then be programmed for the specific room they are assigned to. A customer service agent dedicated to the healthcare facility or a third party customer service representative may then, as needed, contact the listed external contacts to confirm or provision the connections that they want to have during the hospital stay of the admitted patient. Approved network contacts of the patient would be able to send all of the information or services to this patient location that they would be able to do to a private residence location. If the patient is already a user of the system at their residential location, the profile of their home system can be ported to their location in the hospital.

The location and profile of each patient is also available to all staff operating locations. A summary list of patients provides access to that patient via phone, speaker phone, or video conference connection. This structure supports automated electronic access to each patient for doctors, nurses, or other staff that are at a location with a system that supports their access.

Each patient interaction within the healthcare system may be automatically recorded for patient care, billing and liability issues. This recording and other event information may be available for automated updates to the healthcare Electronic Medical Record (EMR) system.

The interactive terminal 1012 in the patient room may be controlled by the same structure as the remote control 1024 that is used in other operating environments. However, the healthcare environment may also have the option of a wired remote that also has the speaker built into the remote control 1024 to create more localized audio.

The nurse or other staff may also use the system and its automated connections to create contact with the patient's outside network or external medical resources.

The video phone connection may be capable of video conference calls with multiple parties at the same time.

The video capabilities of the system provide opportunities for the delivery of education, training, therapy, or exercise video or audio material recommended or required by the healthcare network. A broadband accessible library of video and audio material may be developed and stored for the benefit of the patient as part of the healthcare operation. Video or audio material can be viewed by individual patients on their own schedule and not be limited to a broadcast schedule as is typical on a hospital television system. The video or audio access list of the patient can be populated by customer service to provide automatic access to the recommended material. The system can verify and document the patient use of the required or suggested video or audio material. Video or audio material can also be made available to a patient that has access to an interactive terminal 1012 at home.

Disabled Housing Management Applications

The system as described may also be used to provide utilities and value for organizations that manage disabled persons or other high need populations. For example, housing and service management organizations can operate remotely at times with reduced on-site staff in order to reduce cost and expand private independent living. Remote housing operations have the ability to add security sensors and monitors associated with the interactive terminal 1012 to meet the unique residential monitoring needs of their operations and have those added features hosted on the system structure. Housing management staff may be given full remote access to and control of housing applied systems as needed. Their notification and access may be given priority over the customer service operator. This control can include but not be limited to: (1) security cameras, (2) speaker phone systems, (3) video phone connections, (4) door locks, (5) smoke detectors and other alarm systems, (6) medical sensor data, and (7) lifestyle sensor data.

Housing management may also have the option of providing some central services for their customers. These services could include but not limited to automated printing services for text or photos that are not located in the residence of their customers or managed by them.

Senior Housing Management Applications

Senior housing organizations can range from the management of individual residences, independent living communities, assisted living communities, and nursing homes. Each of these resident environments can provide the personal benefits of the system described to residents and provide different levels of remote residential management and control for the organizations or individuals providing services. The list of implemented services may change depending on the location and availability of support staff and the level of security and monitoring that is desired. One benefit of the system structure described is the ability to extend the time that each resident can have at each level of care.

The nursing home model may be similar to the healthcare application model described above, while the assisted living model may be a combination of the healthcare model and the disabled residential management profile described above. Services to independent living locations may be similar to the remote disabled residential profile.

The system structure described may be used to facilitate the further growth of a caretaker industry where either non-professional (volunteer) or professional (fee based) caretakers provide services for users leveraging the structure of the managed system to provide services to the independent user. These services may include, but are not limited to, (1) private medical and non-medical on-site monitoring, (2) care management, (3) personal buying services, (4) residential maintenance, (5) security, and (6) other applications or utilities.

Since the described system is an application layer that is network agnostic, a user can take the interactive terminal 1012 with them to a new or temporary location where it can be hosted on the available broadband network. This means that if a user moves to a new residential location they can take the interactive terminal 1012 (or portions thereof) with them. It also means that if they own multiple residences that they can have full access to their system and services at any location that has adequate broadband service.

In some embodiments, the system includes the use of voice activated system commands where needed and applicable as an alternative to or in addition to the use of the remote control 1024. Voice commands may be available for various actions including, but not limited to, (1) turning the speaker phone on and answering a call or dialing a connection, (2) making an emergency call, (3) turning the video camera 1020 on or off, (4) returning the host 1016 to the primary screen, and (5) calling customer service.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

We claim:

1. A method for providing content and services to a user, comprising:
receiving, at a user terminal, content from a network entity, wherein access to at least a portion of the content is controllable by a remote system manager;
displaying the content to the user via a user interface;
receiving one or more interactive inputs from the user at the user terminal; and
performing one or more functions associated with the content based on the one or more interactive inputs.

2. The method of claim 1, wherein the content comprises medical content.

3. The method of claim 2, wherein the medical content comprises one or more medical menu options comprising one or more of sending a caregiver message, contacting a caregiver, initiating a medical appointment, taking a medical reading, viewing a personal health record, and ordering medicine.

4. The method of claim 3, wherein taking a medical reading comprises measuring one or more of blood glucose, blood oxygen, blood pressure, heart rate, weight, body mass index, peak expiatory flow rate, and body temperature.

5. The method of claim 1, wherein the content comprises calendar content comprising at least one or more of an appointment display and an option to add one or more events to a calendar.

6. The method of claim 1, wherein the content comprises contact list content.

7. The method of claim 1, wherein the one or more interactive inputs comprise a user selection of a portion of the content.

8. An apparatus for multimedia presentation to a user, comprising:
a content receiving component configured to receive content from a network entity, wherein at least a portion of the content is controllable by a remote system manager;
a display component configured to display the content to the user via a user interface;
an input receiving component configured to receive one or more interactive inputs from the user at the user terminal; and
a user interaction component configured to perform one or more functions associated with the content based on the one or more interactive inputs.

9. The apparatus of claim 8, wherein the content comprises medical content.

10. The apparatus of claim 9, wherein the medical content comprises one or more medical menu options comprising one or more of sending a caregiver message, contacting a caregiver, initiating a medical appointment, taking a medical reading, viewing a personal health record, and ordering medicine.

11. The apparatus of claim 10, further comprising a medical reading component configured to measure one or more of blood glucose, blood oxygen, blood pressure, heart rate, weight, body mass index, peak expiatory flow rate, and body temperature.

12. The apparatus of claim 8, wherein the content comprises calendar content comprising at least one or more of an appointment display and an option to add one or more events to a calendar.

13. The apparatus of claim 8, wherein the content comprises contact list content.

14. The apparatus of claim 8, wherein the one or more interactive inputs comprise a user selection of a portion of the content.

15. A method of controlling content available to a user, comprising:
selecting a user of a remote user terminal from a user list;
setting content permissions defining the content that is accessible to the user; and
saving the content permissions.

16. The method of claim 15, wherein saving the content permissions comprises saving the content permissions to a remote database.

17. The method of claim 15, wherein saving the content permissions comprises saving the content permissions to the remote user terminal.
